(19) United States
(12) Patent Application Publication
(10) Pub. No.: US 2025/0121091 A1
(43) Pub. Date: Apr. 17, 2025

(54) PARTICLES WITH COVALENTLY LIPID-BOUND IMMUNOSTIMULATORY AGENTS

(71) Applicant: Massachusetts Institute of Technology, Cambridge, MA (US)

(72) Inventors: Darrell J. Irvine, Arlington, MA (US); Paula T. Hammond-Cunningham, Newton, MA (US); Ivan Susin Pires, Cambridge, MA (US)

(73) Assignee: Massachusetts Institute of Technology, Cambridge, MA (US)

(21) Appl. No.: 18/801,075

(22) Filed: Aug. 12, 2024

Related U.S. Application Data
(60) Provisional application No. 63/589,927, filed on Oct. 12, 2023.

(51) Int. Cl. A61K 47/69; A61K 9/127; A61K 38/20; A61K 47/54; A61P 35/00

(52) U.S. Cl. CPC A61K 47/6915; A61K 9/1273; A61K 38/208; A61K 47/544; A61P 35/00

(57) ABSTRACT

Disclosed herein are particles, compositions, methods, and kits of particles comprising immunostimulatory agents that can be useful in the treatment or prevention of diseases, such as cancer.

### CROSS-REFERENCE TO RELATED APPLICATIONS

This application claims priority to International patent application, PCT/US2023/076753, filed Oct. 12, 2023, which claims priority under 35 U.S.C. § 119 (e) to U.S. Provisional Patent Application, U.S. Ser. No. 63/589,927, filed Oct. 12, 2023, the contents of each of which are incorporated herein by reference in their entireties.

### GOVERNMENT SUPPORT

This invention was made with government support under AI161297, AI161818, CA235375, and CA274651 awarded by the National Institutes of Health. The government has certain rights in the invention.

### BACKGROUND

Particles such as liposomes, nanoparticles, and lipid nanoparticles have been used to deliver agents to cells. Delivery of certain agents to specific cells is key to developing effective disease treatments. Clinical success of nanoparticles requires development of efficient delivery systems to facilitate such therapies in patients. Accordingly, new particles capable of specifically delivering agents to cells are needed.

### SUMMARY OF THE INVENTION

In one aspect, provided herein is a particle comprising: a liposome, wherein the liposome comprises at least one first lipid covalently bonded to at least one immunostimulatory agent; and the first lipid forms an outer surface of the liposome; a polymer coating, wherein the polymer coating comprises: at least one layer including a polycation, wherein the polycation is non-covalently associated with the outer surface of the liposome; and at least one layer including a polyanion, wherein the polyanion is non-covalently associated with the at least one polycation layer.

In another aspect, the present disclosure provides a pharmaceutical composition comprising a plurality of particles defined herein, and a pharmaceutically acceptable excipient.

In another aspect, provided herein is a particle or a pharmaceutical composition described herein for use in treating a proliferative disease or immune disorder in a subject.

In another aspect, the present disclosure provides use of a particle or a pharmaceutical composition described herein for the manufacture of a medicament for treating proliferative disease or immune disorder in a subject.

In another aspect, provided herein is a method of delivering an immunostimulatory agent to a target cell, wherein the method comprises contacting the target cell with a particle defined herein.

In another aspect, the present disclosure provides a method of recruiting immune cells to a target cell, wherein the method comprises contacting the target cell with a particle described herein.

In another aspect, provided herein is a method of treating a disease in a subject in need thereof, the method comprising administering to the subject an effective amount of a particle described herein.

In another aspect, the present disclosure provides a kit comprising: a particle or a pharmaceutical composition described herein; and instructions for using the particle or the pharmaceutical composition.

### BRIEF DESCRIPTION OF THE DRAWINGS

The accompanying drawings, which constitute a part of this specification, illustrate several embodiments of the invention and together with the description, provide non-limiting examples of the invention. The figures are exemplary and do not limit the scope of the present disclosure.

FIGS. 1A-1I relate to layer-by-layer nanoparticles (LbL-NPs) conjugated with IL-12 via either maleimide-cysteine reaction or nickel-histidine interaction. FIG. 1A shows a schematic for assembly of LbL-IL-12-NPs. FIG. 1B shows a schematic of exemplary linker chemistries used for conjugation of IL-12 onto NPs. FIG. 1C shows intensity-weighted hydrodynamic size (Z-avg), number average size (#-avg), and polydispersity index (PDI) of NPs during synthesis as measured via DLS. FIG. 1D shows Zeta potential of NPs during synthesis as measured via electrophoretic mobility in deionized water. FIG. 1E shows yield and weight loading of IL-12 for unlayered and layered particles with nickel-histidine linker (Ni-UL and Ni-LbL) and unlayered and layered particles with maleimide-cysteine bond (Mal-UL and Mal-LbL). Negative-stain (NS) TEM with phosphotungstic acid of particles during synthesis with nickel-containing lipids are shown in FIG. 1F and those with maleimide-containing lipids are shown in FIG. 1G-scale bars represent 200 nm. FIG. 1H shows confocal microscopy of HM-1 cells after 4 hour incubation with Mal-LbL NPs. FIG. 1I shows confocal microscopy of HM-1 cells after 4 hour incubation with Mal-UL NPs. Data are presented as mean values±s.d with n=3 independent batches of NPs. Statistical comparisons in e performed using two-way analysis of variance (ANOVA) with Tukey's multiple-comparisons test.

FIG. 2A shows the measurement of conjugated IL-12 permeability through 300 kDa centrifugal filters relative to free IL-12 when NPs incubated in 10% FBS and in (FIG. 2B) 100% FBS (mean±s.e.m). FIG. 2C shows total IL-12 detected in Mal-LbL particles via ELISA with particles in either deionized water (DI) or 10% FBS relative to Mal-UL (mean±s.d.). FIG. 2D shows dye-labeled PLR and PLE release kinetics from Mal-LbL NPs (mean±s.e.m). FIG. 2E shows zeta potential of Mal-LbL and Mal-UL NPs in 10 mM HEPES, 15 mM NaCl compared to zeta potential of Mal-LbL NPs exposed to 10 mM HEPES and 150 mM then washed and transferred to 10 mM HEPES and 15 mM NaCl (Mal-LbL*) (mean±s.d.). FIG. 2F shows fraction of Mal NP fluorescence associated with HM-1 cells (ratio between cell-associated fluorescence and free NPs) relative to unlayered (UL) particles for UL NPs, PLR-only NPs and PLR-PLE NPs (mean±s.d.). FIG. 2G shows confocal microscopy of Mal-LbL NPs with fluorescently tagged lipids and PLE after serum treatment for 48 hours and dosed to HM-1 cells for 4 hours. FIG. 2H shows association of NP fluorescence with HM1 cells in vitro relative to unlayered NPs after 24 hours of incubation with either fresh NPs (no incubation) or after incubation in 100% FBS for 48 hours (mean±s.d.). FIG. 2I shows association of IL-12 fluorescence with OV2944-HM1 cells in vitro relative to free IL-12 after 24 hours of incubation with either fresh NPs or after incubation in 100% FBS for 48 hours (mean±s.d.). FIG. 2J shows representative HM-1 cell confocal microscopy dosed with Ni-LbL particles after 48 hours of incubation in 100% serum for 4 hours. FIG. 2K shows representative confocal microscopy of HM-1 cells dosed with Mal-LbL particles after 48 hours of incubation in 100% serum for 4 hours. Data are representative of at least two independent experiments with at least n=2 technical replicates per group. Statistical comparisons in FIG. 2F and FIG. 2I performed using one-way analysis of variance (ANOVA) with Tukey's multiple-comparisons test and in 1 and m two-way ANOVA with Tukey's multiple-comparisons.

FIG. 3A and FIG. 3B show representative confocal microscopy images of HM-1 cells dosed with Mal-LbL NPs with fluorescently tagged PLE (FIG. 3A) and PLR (FIG. 3B) for four hours. FIG. 3C shows confocal microscopy images of HM-1 cells dosed with Mal-LbL NPs with fluorescently tagged PLR after incubation with 100% FBS for 48 hours then allowed to interact with HM-1 cells for four hours.

FIG. 4A shows representative confocal images of Mal-LbL NPs dosed to HM-1 cells for 4 hours. FIG. 4B shows representative confocal images of Ni-LbL NPs dosed to HM-1 cells for 4 hours. FIG. 4C and FIG. 4D show representative confocal image of HM-1 cells dosed with Ni-UL particles for 4 hours (FIG. 4C) prior or (FIG. 4D) after serum incubation for 48 hours.

FIG. 4E and FIG. 4F Representative confocal image of HM-1 cells dosed with Mal-UL particles for 4 hours (FIG. 4E) prior or (FIG. 4F) after scrum incubation for 48 hours. FIG. 4G shows representative confocal image of HM-1 cells incubated with IL-12 for 24 hours. FIG. 4H shows representative confocal image of HM-1 cells incubated with Mal-LbL NPs for 24 hours after incubation in 100% serum for 48 hours.

FIG. 5A shows schematic of protocol for assessment of NP retention. FIG. 5B shows representative confocal images of HM-1 cells treated with NPs indicated based on protocol shown in FIG. 5A.

FIG. 6A shows measurement of fluorescently-tagged lipid permeability through 300 kDa centrifugal filters relative to free IL-12 when incubated in 10% FBS and in (FIG. 6B) 100% FBS (mean±s.e.m). FIG. 6C shows confocal microscopy image of HM-1 cells dosed with lipid-lipid FRET Mal-LbL NPs to assess NP stability via acceptor photobleaching. FIG. 6D shows schematic of high-density lipid-lipid FRET pair used to assess NP stability. FIG. 6E shows quantification of NP fluorescence from donor or acceptor fluorophore in DI (NP form) or in DMSO (dissociated lipids) (mean±s.d.). FIG. 6F shows confocal microscopy of HM-1 cells dosed with high density lipid-lipid FRET Mal-LbL NPs for four hours with FRET efficiency measured via acceptor photobleaching. FIG. 6G shows diagram depicting mechanism of Mal-LbL NP presentation of IL-12 via either surface-bound protein or via lipid exchange of lipid-conjugated IL-12. Data are representative of at least two independent experiments with at least n=2 technical replicates per group.

FIG. 7A shows IL-12 bioactivity curves measured via a HEK-Blue IL-12 cell assay. FIG. 7B shows calculated IL-12 EC50 from HEK-Blue IL 12 cell assay response (mean±s.e.m.). IL-12 bioactivity curves of Mal-UL and Mal-LbL in the (FIG. 7C) retentate or (FIG. 7D) permeate of 300 kDa centrifugal filters after 48 hours of incubation in 100% FBS.

FIG. 8A shows analysis of a healthy mouse compared to a 1-week post-HM-1 inoculated mouse via micro computed tomography (micro-CT) scan after dosing i.p. with Omnipaue contrast agent. FIG. 8B HM-1 tumor BL1 signal in organs compared to healthy tissue (n=11) two weeks after tumor inoculation (n=34). FIG. 8C bioluminescence readings of intestine, lung, omentum and upper genital tract (UGT) of 1-week post-HM-1-luc inoculation. Statistical comparisons in b performed using nonparametric Mean-Whitney test with correction for multiple comparison based on a false discovery rate of 1%.

FIG. 9A shows schematic for the generation of NPs with both lipid and IL-12 fluorescent tags. FIG. 9B shows treatment schedule for pharmacokinetic and biodistribution analysis. FIG. 9C shows in vivo mice i.p. NP radiant efficiency (mean±s.e.m.). FIG. 9D shows area under the curve quantification of in vivo mice i.p. NP radiant efficiency (mean±s.e.m.). FIG. 9E shows in vivo mice i.p. IL-12 radiant efficiency (mean±s.e.m.). FIG. 9F shows area under the curve quantification of in vivo mice i.p. IL-12 radiant efficiency (mean±s.e.m.). FIG. 9G and FIG. 9G show total NP radiant efficiency from ex vivo organs (FIG. 9G) 4 hours (n=4; mean±s.e.m.) and (FIG. 9H) 24 hours (n=5; mean±s.e.m.) after dosing. FIG. 9I and FIG. 9J show total IL-12 radiant efficiency from ex vivo organs (FIG. 9I) 4 hours (n=4; mean±s.e.m.) and (FIG. 9J) 24 hours (n=5; mean±s.e.m.) after dosing. Statistical comparisons performed using two-way analysis of variance (ANOVA) with Tukey's multiple-comparisons.

FIG. 10 shows biodistribution of IL-12 constructs. IVIS bioluminescence and fluorescence images for mice bearing two week old i.p. HM-1-luc tumors dosed with IL-12 constructs. Organs included in the analysis were liver, spleen, heart, digestive tract, kidney, UGT, and omentum.

FIG. 11A-E show cryogenic omentum tumor sections of two-week-old HM-1-luc mice 24 hours after dosing with (FIG. 11A) free IL-12, (FIG. 11B) Ni-UL, (FIG. 11C) Mal-UL, (FIG. 11D) Ni-LbL, (FIG. 11E) Mal-LbL.

FIGS. 12A-12C show confocal microscopy with increasing magnification for Mal-LbL NPs-exposed omentum tissue. FIG. 12A shows stitched images captured with a 30× objective lens. Confocal image inside tumor tissue with (FIG. 12B) 60× lens and (FIG. 12C) 100× lens. White arrows point to NP depots on the outside of the cell membrane.

FIG. 13 shows bi-weekly i.p. dosing for two weeks of 20 μg of IL-12 or Mal NPs docs not cause systemic toxicity. Panel A shows healthy mouse bodyweight changes upon dosing of dextrose or 20 μg of Mal-LbL at day 0 and day 6.

FIGS. 14A-14H shows characterization of Mal IL-12 NPs. FIG. 14A shows intensity-weighted hydrodynamic size (Z-avg), number average size (#-avg), and PDI of NPs during synthesis as measured via DLS (mean±s.d.). FIG. 14B shows zeta potential of NPs during synthesis as measured via electrophoretic mobility in deionized water (mean±s.d.). FIG. 14C shows association of NP fluorescence with HM1 cells in vitro relative to unlayered NPs after 4 and 24 hours of incubation with either fresh NPs (no incubation) or after incubation in 100% FBS for 48 hours. FIG. 14D shows calculated IL-12 EC50 of IL-12 compared to SAT-LbL NPs from HEK-Blue IL-12 assay (mean±s.e.m.). FIG. 14E shows normalized FRET efficiency of unsaturated (UNsat) NPs and SAT NPs in either UL or LbL formats when incubated in either 10% or 100% FBS. FIG. 14F shows FRET efficiency of NPs. FIG. 14G shows confocal microscopy of HM-1 cells incubated with SAT-UL NPs for 4 hours. FIG. 14H shows confocal microscopy of HM-1 cells incubated with SAT-LbL NPs for 4 hours. All FRET NPs were made with a high-density of fluorophores. Statistical comparisons performed using two-way analysis of variance (ANOVA) with Tukey's multiple-comparisons.

FIG. 15A shows dosing scheme for two weekly doses. FIG. 15B shows IFN-γ ELISpot from white blood cells one month after start of study. FIG. 15C shows in vivo bioluminescence readings of mice bearing HM-1-luc tumors treated at day 7 and day 14 with IL-12 constructs. FIG. 15D shows s curves for mice bearing HM-1-luc tumors treated at day 7 and day 14 with IL-12 constructs. FIG. 15E shows in vivo bioluminescence readings of mice bearing HM-1-luc tumors treated at day 7 with 20 μg of IL-12 constructs or equivalent amount of carrier LbL-NP (unloaded-LbL). FIG. 15F shows survival curves for mice bearing HM-1-luc tumors treated at day 7 with 20 μg of IL-12 constructs or equivalent amount of carrier LbL-NP (unloaded-LbL). Statistical comparisons for b performed using one-way analysis of variance (ANOVA) with Tukey's multiple-comparisons. Statistical comparisons between survival curves were performed using a log-rank (Mantel-Cox) test.

FIG. 16A shows treatment scheme for two weekly doses combined with immune checkpoint inhibitors. FIG. 16B shows IFN-γ ELISpot from white blood cells one month after start of study. FIG. 16C shows in vivo bioluminescence readings and (FIG. 16D) survival curves of mice bearing HM-1-luc tumors treated at day 7 and day 14 with IL-12 constructs and at day 8 and 15 with checkpoint inhibitors. Statistical comparisons performed using one-way analysis of variance (ANOVA) with Tukey's multiple-comparisons. Statistical comparisons between survival curves were performed using a log-rank (Mantel-Cox) test.

FIG. 17 shows a schematic where IL-12 is bound to a lipid by nickel: histidine and maleimide-cysteine linkages

FIG. 18 shows schematics of control, free IL-12, and various particles. experimental groups where UL stands for unlayered and LbL stands for layer-by-layer.

FIG. 19A shows graphs depicting IL-12 LbL-NPs size and polydispersity. FIG. 19B shows a plot depicting zeta potential for the surface charge. FIG. 19C shows a dose-response cure for various particles bioactivity.

FIG. 20A shows the biodistribution of particles after 24 hours. FIG. 20B shows the total radiance efficiency for various particles.

FIG. 21 shows ELISPOT assay depicting increased tumor retention of IL-12 resulting in increased anti-tumor specific immune response from weekly dosing.

FIG. 22A shows the probability of survival of mice dosed with various particles. FIG. 22B shows the probability of survival of mice dosed with various particles and/or checkpoint inhibitors.

### DEFINITIONS

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles (e.g., lipid nanoparticles), macroscopic particles, pellets, crystals, aggregates, composites, pulverized, milled or otherwise disrupted matrices, and cross-linked protein or polysaccharide particles. A particle may be composed of a single substance or multiple substances. In certain embodiments, the particle is a viral particle. In other embodiments, the particle is a liposome. In certain embodiments, the particle is a micelle. In certain embodiments, the particle is not a viral particle. In certain embodiments, the particle is not a micelle. In certain embodiments, the particle is substantially solid throughout. In certain embodiments, the particle is a nanoparticle. In certain embodiments, the particle is a microparticle.

The term “nanoparticle” refers to a particle having an average (e.g., mean) dimension (e.g., diameter) of between about 1 nanometer (nm) and about 1 micrometer (μm) (e.g., between about 1 nm and about 300 nm, between about 1 nm and about 100 nm, between about 1 nm and about 30 nm, between about 1 nm and about 10 nm, or between about 1 nm and about 3 nm), inclusive.

The term “microparticle” refers to a particle having an average (e.g., mean) dimension (e.g., diameter) of between about 1 micrometer (μm) and about 1 millimeter (mm) (e.g., between about 1 μm and about 100 μm, between about 1 μm and about 30 μm, between about 1 μm and about 10 μm, or between about 1 μm and about 3 μm), inclusive.

The term “polymer” refers to a compound comprising two or more covalently connected repeating units. In certain embodiments, a polymer is naturally occurring. In certain embodiments, a polymer is synthetic (i.e., not naturally occurring).

The term “polyelectrolyte”, as used herein refers to a polymer which under a particular set of conditions (e.g., physiological conditions) has a net positive or negative charge. In some embodiment, a polyelectrolyte is or comprises a polycation; in some embodiments, a polyelectrolyte is or comprises a polyanion. Polycations have a net positive charge and polyanions have a net negative charge. The net charge of a given polyelectrolyte may depend on the surrounding chemical conditions, e.g., on the pH. Exemplary polyelectrolytes for use in polymeric coatings in the composition disclosed herein are but not limited to: poly(L-arginine) (PLR), poly-L-lysine (PLL), polyethylenimine (PEI), poly(β-amino esters), poly-L-glutamic acid (PLE), polyarginine, polyglutamic acid, polylysine, heparin folate, heparin sulfate, fucoidan, sulfated-β-cyclodextrin, hyaluronic acid (HA), polyglutamic acid-block-polyethylene glycol, poly-L-aspartic acid (PLD), polyaspartic acid, polystyrene sulfonate (SPS), polyacrylic acid (PAA), linear poly(ethylene imine) (LPEI), poly(diallyldimethyl ammonium chloride) (PDAC), polyallylamine hydrochloride (PAH), poly(L-lactide-co-L-lysine), polyserine ester, poly (4-hydroxy-L-proline ester), poly [α-(4-aminobutyl)-L-glycolic acid], sodium polystyrene sulfonate, dextran sulfate (DXS), alginate, and chondroitin sulfate.

The term “cation” or “cationic” used herein refer to a species which has a net positive charge. The term “anion” or “anionic” used herein refer to a species which has a net negative charge.

The term “sterol” refers to a subgroup of steroids also known as steroid alcohols, i.e., a steroid containing at least one hydroxyl group. Sterols are usually divided into two classes: (1) plant sterols also known as “phytosterols,” and (2) animal sterols also known as “zoosterols.” The term “sterol” includes, but is not limited to, cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers.

As used herein, the term “associated with” refers to a direct association between two molecules, due to, for example, covalent, electrostatic hydrophobic, and ionic and/or hydrogen-bond interactions.

As used herein, the term “affinity” refers to the equilibrium constant for the reversible binding of two agents (e.g., tag and a ligand, or a metal ion and a ligand) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of a ligand for unrelated peptides or compounds. Affinity of a ligand to its binding partner can be, for example, from about 100 nanomolar (nM) to about 0.1 nM, from about 100 nM to about 1 picomolar (pM), or from about 100 nM to about 1 femtomolar (fM) or more. An “affinity ligand” is a ligand having affinity for a binding partner.

The term “unsaturated” or “unsaturated bond” (e.g., unsaturated carbon-carbon bond) refers to a double or triple bond.

The term “saturated” or “fully saturated” refers to a moiety that does not contain a double or triple bond, e.g., the moiety only contains single bonds.

Herein, the term “linker” (also known as “linker molecules” or “cross-linkers” or “spacers”) are molecules which may be used to conjugate one atom to another in a composition. The majority of known linkers react with amine, carboxyl, and sulfhydryl groups. Linker molecules may be responsible for different properties of the composition. The length of the linker should be considered in light of molecular flexibility during the conjugation step, and the availability of the conjugated molecule for its target. Longer linkers may this improve the biological activity of the compositions as well as the case of preparation of them. The geometry of the linker may be used to orient a molecule for optimal reaction with a target. A linker with flexible geometry may allow the entire composition to conformational adapt as it binds a target sequence. The nature of the linker may be controlled by the monomeric units along with the polymer, e.g., a block polymer in which there is a block of hydrophobic monomers interspersed with a block of hydrophilic monomers.

The chemistry of preparing and utilizing a wide variety of molecular linkers is well-known in the art and many premade linkers for us in conjugating molecules are commercially available from vendors such as Pierce Chemical Co., Roche Molecular Biochemicals, United States Biological.

Exemplary linker molecules for use in the compositions of the invention include, but are not limited to: aminocaproic acid (ACA); polyglycine, and any other amino acid polymer polymers such as polyethylene glycol (PEG), polymethylmethacrylate (PMMA), polypropylene glycol (PPG); homobifunctional reagents such as APG, AEDP, BASED, BMB, BMDB, BMH, BMOE, BM [PEO] 3, BM [PEO] 4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS; heterobifunctional reagents such as ABH, AEDP, AMAS, ANB-NOS, APDP, ASBA, BMPA, BMPH, BMPS, EDC, EMCA, EMCH, EMCS, KMUA, KMUH, GMBS, Glossary LC-SMCC, LC-SPDP, MBS, MBUS, M2C2H, MPBH, MSA, NHS-ASA, PDPH, PMPI, SADP, SAED SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS. residues). Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS; and trifunctional linkers such as Sulfo-SBED.

Branched linkers may be prepared Branched linkers may be prepared or used so that multiple moieties per linker are able to react. Such multiply reactive linkers allow the creation of multimeric binding sites.

The term “pKa,” as used herein, includes the negative decadic logarithm of the ionization constant (Ka) of an acid; equal to the pH value at which equal concentrations of the acid and conjugate base forms of a substance (often a buffer) are present.

The term “hydrophobic,” as used herein, refers to a compound that has an octanol/water partition coefficient (K_{ow}) greater than about 10 at about 23° C.

The term “hydrophilic,” as used herein, refers to a compound that has an octanol/water partition coefficient (K_{ow}) less than about 10 at about 23° C.

As used here, the term “PEG-lipid” refers to a PEGylated lipid.

An “amino acid” refers to natural and unnatural D/L alpha-amino acids, as well as natural and unnatural beta- and gamma-amino acids. A “peptide” refers to two amino acids joined by a peptide bond. A “polypeptide” refers to three or more amino acids joined by peptide bonds. An “amino acid side chain” refers to the group(s) pended to the alpha carbon (if an alpha amino acid), alpha and beta carbon (if a beta amino acid), or the alpha, beta, and gamma carbon (if a gamma amino acid).

A “protein,” “peptide,” or “polypeptide” comprises a polymer of amino acid residues linked together by peptide bonds. The term, as used herein, refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

A cytokine is a signaling protein regulating biological functions such as innate and acquired immunity, hematopoiesis, inflammation and repair, and proliferation through mostly extracellular signaling. Interleukin 12 (Il-12 or IL-12) is a cytokine that is naturally produced by dendritic cells, macrophages, neutrophils, and human B-lymphoblastoid cells in response to antigenic stimulation. Il-12 is composed of a bundle of four alpha helices. It is a heterodimeric cytokine encoded by two separate genes, Il-12A and Il-12B. Single chain interleukin 12 (scIL-12) is a protein in which the subunits of heterodimeric IL-12 are covalently bonded together. For example, interleukin 12 can be monomerized by introduction of a peptide linker between the subunits of heterodimeric cytokine. scIL-12 may be a fusion protein.

The term “endocytosis” or “endocytosed” used herein refers to a cellular process known to those skilled in the art, wherein a substance is internalized by a cell.

The terms “composition” and “formulation” are used interchangeably.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “biological component” refers to any substance in the biological sample or the in vivo biological environment (e.g., proteins, lipids, antibodies, RNAs, DNAs, small molecules, enzymes, serum component, tumor-replated component etc.).

The term “target tissue” refers to any biological tissue of a subject (including a group of cells, a body part, or an organ) or a part thereof, including blood and/or lymph vessels, which is the object to which a compound, particle, and/or composition of the present disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

A “target cell” refers to a cell in a subject (in vivo) or ex vivo to which a compound, particle, and/or composition of the present disclosure is delivered. A target cell may be an abnormal or unhealthy, which may need to be treated. A target cell may also be a normal or healthy but is under a higher-than-normal risk of becoming abnormal or unhealthy, which may need to be prevented. In certain embodiments, the target cell is a cancer cell. In certain embodiments, the target cell is an ovarian cancer cell (e.g., HM-1 cell). In certain embodiments, the target cell is a colon cancer cell. In certain embodiments, the target cell is a brain cancer cell. In certain embodiments, the target cell is a skin cancer cell. In certain embodiments, the target cell is a head and neck cancer cell. In certain embodiments, the target cell is a lung cancer cell.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a particle described herein, or a composition thereof, in or on a subject.

The term “intravenous injection” is used herein to refer to a mode of administration wherein the composition is administered through an injection into the vein or veins.

The term “intraperitoneal injection” is used herein to refer to a mode of administration wherein the composition is administered through an injection into the peritoneum.

The term “oral intake” is used herein to refer to a mode of administration wherein the composition is administered thought the mouth and swallowed.

The term “inhalation” is used herein to refer to a mode of administration wherein the composition is administered thought the mouth or nose by inhaling.

The term “intratumoral injection” is used herein to refer to a mode of administration wherein the composition is administered through an injection into a tumor.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

An “effective amount” of a particle described herein refers to an amount sufficient to elicit the desired biological response. An effective amount of a particle described herein may vary depending on such factors as the desired biological endpoint, severity of side effects, disease, or disorder, the identity, pharmacokinetics, and pharmacodynamics of the particular particle, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a particle described herein in a single dose. In certain embodiments, an effective amount is the combined amounts of a particle described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a particle for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a particle per unit dosage form.

In certain embodiments, the particles of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The present disclosure provides pharmaceutical compositions comprising a particle disclosed herein, or a pharmaceutically acceptable salt thereof, and optionally a pharmaceutically acceptable excipient. In certain embodiments, the pharmaceutical composition described herein comprises a particle of disclosed herein, or a pharmaceutically acceptable salt thereof, and a pharmaceutically acceptable excipient.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

A “therapeutically effective amount” of a particle or agent described herein is an amount sufficient to provide a therapeutic benefit in the treatment of a condition or to delay or minimize one or more symptoms associated with the condition. A therapeutically effective amount of a particle or agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for delivering an agent to a subject or a cell. In certain embodiments, a therapeutically effective amount is an amount sufficient for delivering a polynucleotide to a subject or a cell. In certain embodiments, a therapeutically effective amount is an amount sufficient for treating a disease, disorder, or condition. In certain embodiments, a therapeutically effective amount is an amount sufficient for delivering an agent to a subject or a cell and treating a disease, disorder, or condition. In certain embodiments, a therapeutically effective amount is an amount sufficient for delivering a polynucleotide to a subject or a cell and treating a disease, disorder, or condition. In certain embodiments, a therapeutically effective amount is an amount sufficient for delivering mRNA to a subject or a cell and treating a disease, disorder, or condition.

A “prophylactically effective amount” of a particle or agent described herein is an amount sufficient to prevent a condition, or one or more symptoms associated with the condition or prevent its recurrence. A prophylactically effective amount of a particle or agent means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for delivering an agent to a subject or a cell. In certain embodiments, a prophylactically effective amount is an amount sufficient for delivering a polynucleotide to a subject or a cell. In certain embodiments, a prophylactically effective amount is an amount sufficient for delivering mRNA to a subject or a cell. In certain embodiments, a prophylactically effective amount is an amount sufficient for preventing a disease, disorder, or condition. In certain embodiments, a prophylactically effective amount is an amount sufficient for delivering an agent to a subject or a cell and preventing a disease, disorder, or condition. In certain embodiments, a prophylactically effective amount is an amount sufficient for delivering a polynucleotide to a subject or a cell and preventing a disease, disorder, or condition. In certain embodiments, a prophylactically effective amount is an amount sufficient for delivering mRNA to a subject or a cell and preventing a disease, disorder, or condition.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### Diseases, Disorders, and Conditions

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypercosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; parancoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In certain embodiments, the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, cosinophilic gastrointestinal disorders (e.g., cosinophilic esophagitis, cosinophilic gastritis, cosinophilic gastroenteritis, cosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD). In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

Immune disorders, such as auto-immune disorders, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anacmias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, cosinophilic disease, gastrointestinal disorder (e.g., selected from peptic ulcers, regional enteritis, diverticulitis, gastrointestinal bleeding, cosinophilic gastrointestinal disorders (e.g., cosinophilic esophagitis, cosinophilic gastritis, cosinophilic gastroenteritis, cosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); cosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In certain embodiments, the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, cosinophilic gastrointestinal disorders (e.g., cosinophilic esophagitis, cosinophilic gastritis, cosinophilic gastroenteritis, cosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

### Pharmaceutical Compositions, Kits, and Administration

The present disclosure provides pharmaceutical compositions comprising a particle disclosed herein and a pharmaceutically acceptable excipient.

In certain embodiments, the particle described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a painful condition in a subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, neurological disease, painful condition, psychiatric disorder, or metabolic disorder) in a subject in need thereof. In certain embodiments, the effective amount is an amount effective for recruiting immune molecules to a target cell. In certain embodiments, the effective amount is an amount effective for stimulating am immune response towards a target cell.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present ex vivo.

Pharmaceutical compositions described herein can be prepared by any method known in the art of pharmaceutics. In general, such preparatory methods include bringing the particle described herein into association with a carrier or excipient, and/or one or more other accessory ingredients, and then, if necessary and/or desirable, shaping, and/or packaging the product into a desired single- or multi-dose unit.

Pharmaceutically acceptable carriers can vary in a formulation described herein, depending on the administration route. The formulations described herein can be delivered via any administration mode known to a skilled practitioner. For example, the formulations described herein can be delivered in a systemic manner, via administration routes such as, but not limited to, oral, and parenteral, including intravenous, intramuscular, intraperitoneal, intradermal, and subcutaneous. In some embodiments, the formulations described herein are in a form that is suitable for injection. In other embodiments, the formulations described herein are formulated for oral administration.

When administering parenterally, a formulation described herein can be generally formulated in a unit dosage injectable form (solution, suspension, emulsion). The formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g., PBS). Where necessary, the composition may also include a solubilizing agent and a local anesthetic such as lignocaine to case pain at the site of the injection.

The formulations can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to formulations described herein, however, any vehicle, diluent, or additive used should have to be biocompatible with the active agents described herein. Those skilled in the art will recognize that the components of the formulations should be selected to be biocompatible with respect to the active agent. For in vivo administration, the formulations described herein can be administered with a delivery device, e.g., a syringe. Accordingly, an additional aspect described herein provides for delivery devices comprising at least one chamber with an outlet, wherein the at least one chamber comprises a pre-determined amount of any formulation described herein and the outlet provides an exit for the formulation enclosed inside the chamber. In some embodiments, a delivery device described herein can further comprise an actuator to control release of the formulation through the outlet. Such delivery device can be any device to facilitate the administration of any formulation described herein to a subject, e.g., a syringe, a dry powder injector, a nasal spray, a nebulizer, or an implant such as a microchip, e.g., for sustained-release or controlled release of any formulation described herein.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents or fillers, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents or fillers include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, starches (such as dry starch, cornstarch), sugars (such as powdered sugar), calcium trisulfate, carboxymethylcellulose calcium, dextrate, dextrin, dextrose, fructose, lactitol, lactose, magnesium carbonate, magnesium, maltitol, maltodextrin, maltose, sucrose, glucose, mannitol, silicic acid, xylitol, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary disintegrating agents or disintegrants include agar, algin, alginic acid, sodium alginate, silicates, sodium carbonate, calcium carbonate, carboxymethylcellulose, cellulose, clay, colloidal silicon dioxide, croscarmellose sodium, crospovidone, rubber, magnesium silicate, methylcellulose, potassium krillin, hydroxypropylcellulose (e.g., low substituted Hydroxypropylcellulose), crosslinked polyvinylpyrrolidone, hydroxypropylcellulose, and starch (e.g., sodium glycolate starch, potato or tapioca starch).

Exemplary binding agents include starch (e.g., glycolate starch, cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include agar, ethyl oleate, ethyl laurate, glycerin, blyceryl palmitostearate, magnesium oxide, magnesium stearate, mannitol, poloxamer, glycol, sodium stearyl, sorbitol, zinc stearate, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

In some embodiments, injectable preparations of the compositions disclosed herein are in the form of a ready-to-use (“RTU”) preparation that can be directly administered to a subject. In some embodiments, the RTU preparation is a suspension. In some embodiments, the RTU preparation is a solution. In some embodiments, the RTU preparation is an emulsion. In some embodiments, injectable preparations of the compositions disclosed herein are in the form of a solid that is reconstituted prior to administration. In some embodiments, the solid is a lyophilized solid. In some embodiments, injectable preparations of the compositions disclosed herein are in the form of a liquid or suspension that is diluted prior to administration.

In some embodiments, the pharmaceutical compositions disclosed herein comprise a bulking agent. Bulking agents can be used, e.g., to improve the appearance of a solid composition, to provide visible “bulk” to demonstrate product quality or to facilitate preparation, e.g., of a solid composition prepared for reconstitution prior to administration. Bulking agents can be used for low dose (high potency) drugs that do not have the necessary bulk to support their own structure or provide a visible composition in a unit dosage form. Bulking agents are used in lyophilized formulations. Bulking agents provide a desirable structure for a lyophilized cake comprising pores that provide the means for vapor to escape from the product during lyophilization cycles, and facilitate dissolution on reconstitution. In some embodiments, the bulking agent is mannitol, lactose, sucrose, dextran, trehalose, povidone, dextran, glycine, isoleucine, methionine, or a cyclodextrin (e.g., (2-hydroxypropyl)-β-cyclodextrin).

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (c) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a particle described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the particle in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Particles provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The particles and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the particle or pharmaceutical composition described herein is suitable for topical administration to the eye of a subject.

The exact amount of a particle required to achieve an effective amount will vary from subject to subject, depending, for example, on species, age, and general condition of a subject, severity of the side effects or disorder, identity of the particular particle, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a particle described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a particle described herein. In certain embodiments, a dose described herein includes independently between 1 mg and 3 mg, inclusive, of a particle described herein. In certain embodiments, a dose described herein includes independently between 3 mg and 10 mg, inclusive, of a particle described herein. In certain embodiments, a dose described herein includes independently between 10 mg and 30 mg, inclusive, of a particle described herein. In certain embodiments, a dose described herein includes independently between 30 mg and 100 mg, inclusive, of a particle described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A particle or composition, as described herein, can be administered in combination with one or more additional pharmaceutical agents (e.g., therapeutically and/or prophylactically active agents). The particle or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a particle described herein and an additional pharmaceutical agent shows a synergistic effect that is absent in a pharmaceutical composition including one of the particle and the additional pharmaceutical agent, but not both. In some embodiments, the additional pharmaceutical agent achieves a desired effect for the same disorder. In some embodiments, the additional pharmaceutical agent achieves different effects.

The particle or composition can be administered concurrently with, prior to, or subsequent to one or more additional pharmaceutical agents, which may be useful as, e.g., combination therapies (e.g., chemotherapy, targeted therapy, gene therapy, immune therapy, and hormone therapy). Pharmaceutical agents include therapeutically active agents Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the particle or composition described herein in a single dose or composition or administered separately in different doses or compositions. The particular combination to employ in a regimen will take into account compatibility of the particle described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the particle described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

Anti-cancer agents encompass biotherapeutic anti-cancer agents as well as chemotherapeutic agents. Exemplary biotherapeutic anti-cancer agents include, but are not limited to, immune therapies such as interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab), anti-PD1 (pembrolizumab, nivolumab, cemiplimab), anti-PDL1, anti-CTLA4 (ipilimumab, tremelimumab), anti-TIM-3, anti-LAG-3, anti-NKG2A, anti-CD73, anti-A2aR, anti-B7-H3, and anti-B7-H4).

Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., ‘2’-paclitaxel methyl 2-glucopyranosyl succinate; docetaxel, taxol), cpipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), ncratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoc) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Also encompassed by the disclosure are kits (e.g., pharmaceutical packs). The kits provided may comprise a pharmaceutical composition or particle described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or particle described herein. In some embodiments, the pharmaceutical composition or particle described herein provided in the first container and the second container are combined to form one unit dosage form.

Thus, in one aspect, provided are kits including a first container comprising a particle or pharmaceutical composition described herein. In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a target in a subject or cell. A kit described herein may include one or more additional pharmaceutical agents described herein as a separate composition.

### DETAILED DESCRIPTION OF CERTAIN EMBODIMENTS

Immunotherapy is a promising approach to the treatment of diseases, including proliferative diseases such as cancer. For example, checkpoint inhibitors (CPI) such as anti-PD-1 and anti-PD-L1 can elicit durable complete responses in a proportion of patients suffering from cancer and have been approved across a range of solid tumor indications.¹ However, issues arise when the cancer is late-stage and or is particularly immunosuppressive. For example, ovarian cancer (OC) is often diagnosed when metastatic spread into visceral organs of the peritoneal cavity has already occurred. CPI has not been effective in OC, a finding which is thought to be at least partly due to a highly immunosuppressive nature of the ovarian cancer tumor microenvironment (TME).²⁻⁴

Unfortunately, while there have been extensive efforts to develop therapies for OC, little has changed in its treatment.⁵ Current strategies to treat OC rely primarily on surgery and systemic chemotherapy, which provides an initial good response but is often followed by recurrence with chemoresistance.⁵ Targeted therapies such as antiangiogenic antibodies, poly (ADP-ribose) polymerase (PARP) inhibitors, and folate-targeted antibody drug conjugates (ADCs) have been approved but without substantial improvements in survival.^{5,6} Therefore, new treatment strategies that can overcome the difficulties associated with the treatment of proliferative disease (such as metastatic spread and immunosuppression in cancers) whilst enabling increased survival are urgently needed.

Immunostimulatory agents such as cytokines and costimulatory antibodies may have the potential to overturn the immunosuppressive TME of OC, but systemic and untargeted administration of these therapeutics often leads to dose-limiting toxicities.^{7,8} Thus, much effort has focused on strategies to deliver immunostimulatory agents in a site-specific manner to the TME and induce immune activation while avoiding off-target effects and dose-limiting toxicities seen previously.⁷

The use of particles to target cancer cells by altering their surface chemistries with polyelectrolyte films via layer-by-layer (LbL) deposition was previously reported.⁹ It was shown, however that these compositions resulted in endocytosis or degradation of the particle, thus dampening any therapeutic effect.⁹⁻²⁸

The present disclosure details the mechanistic characterization of particles capable of delivering immunostimulatory agents to a target cell. The inventors have unexpectedly discovered that anchoring immunostimulatory agents such as cytokines to the surface of a particle via a covalent bond improved trafficking and retention of the immunostimulatory agent in tumors compared to non-covalently associated agents, ultimately extending survival compared to free immunostimulatory agents and synergizing with immune checkpoint inhibitors (CPI) leading to improved treatments (including curative treatments and rechallenge remission). Without wishing to be bound by theory, the inventors posit that these improved effects may be due to the trafficking of covalently-bound immunostimulatory agents from particles via lipid-exchange either to serum components or to target cell membranes.

In one aspect, provided herein is a particle comprising:
(a) a liposome, wherein the liposome comprises at least one first lipid covalently bonded to at least one immunostimulatory agent; and the first lipid forms an outer surface of the liposome; and
(b) a polymer coating, wherein the polymer coating comprises:
   at least one layer including a polycation, wherein the polycation is non-covalently associated with the outer surface of the liposome; and
   at least one layer including a polyanion, wherein the polyanion is non-covalently associated with the at least one polycation layer.

In some embodiments, the particle is a nanoparticle.

In certain embodiments, the liposome further comprises a second lipid. In some embodiments, the second lipid is a phospholipid. In certain embodiments, the phospholipid is selected from the group consisting of 1,2-distearoyl-sn-glycero-3-phosphocholine (DSPC), hydrogenated soy phosphatidylcholine (HSPC), 1,2-dipalmitoyl-sn-glycero-3-phosphocholine (DPPC), 1,2-distearoyl-sn-glycero-3-phosphoethanolamine (DSPE), 1,2-dioleoyl-sn-glycero-3-phosphoethanolamine (DOPE), 1,2-Dipalmitoyl-sn-glycero-3-phosphoethanolamine (DPPE), soy phosphatidylcholine, and egg phosphatidylcholine.

In some embodiments, the phospholipid is DSPC. In some embodiments, the phospholipid is HSPC. In some embodiments, the phospholipid is DPPC. In some embodiments, the phospholipid is DSPE. In some embodiments, the phospholipid is DOPE. In some embodiments, the phospholipid is DPPE. In some embodiments, the phospholipid is soy phosphatidylcholine. In some embodiments, the phospholipid is egg phosphatidylcholine.

In some embodiments, the liposome further comprises a third lipid. In some embodiments, the third lipid is a sterol. In some embodiments, the sterol is selected from the group consisting of campesterol, desmosterol, stigmasterol, lanosterol, and sitosterol. In certain embodiments, the sterol is cholesterol. In certain embodiments, the sterol is sitosterol. In certain embodiments, the sterol is campesterol. In certain embodiments, the sterol is stigmasterol. In certain embodiments, the sterol is brassicasterol. In certain embodiments, the sterol is desmosterol. In certain embodiments, the sterol is chalinosterol. In certain embodiments, the sterol is poriferasterol. In certain embodiments, the sterol is clionasterol. In certain embodiments, the sterol is ergosterol. In certain embodiments, the sterol is coprosterol. In certain embodiments, the sterol is codisterol. In certain embodiments, the sterol is isofucosterol. In certain embodiments, the sterol is fucosterol. In certain embodiments, the sterol is clerosterol. In certain embodiments, the sterol is nervisterol. In certain embodiments, the sterol is lathosterol. In certain embodiments, the sterol is stellasterol. In certain embodiments, the sterol is spinasterol. In certain embodiments, the sterol is chondrillasterol. In certain embodiments, the sterol is peposterol. In certain embodiments, the sterol is avenasterol. In certain embodiments, the sterol is isoavenasterol. In certain embodiments, the sterol is fecosterol. In certain embodiments, the sterol is pollinastasterol. In certain embodiments, the sterol is all natural or synthesized forms and derivatives thereof, including isomers.

In some embodiments, the liposome further comprises a fourth lipid; and the fourth lipid is an anionic lipid. In some embodiments, the anionic lipid is selected from the group consisting of 1-palmitoyl-2-olcoyl-sn-glycero-3-phospho-(1′-rac-glycerol) (POPG), 1,2-distearoyl-sn-glycero-3-phosphoglycerol (DSPG), 1,2-dipalmitoyl-sn-glycero-3-phosphoglycerol (DPPG), 1,2-diolcoyl-sn-glycero-3-phospho-rac-(l′-glycerol) (DOPG), 1-palmitoyl-2-oleoyl-sn-glycero-3-phosphoinositol (POPI), 1,2-distearoyl-sn-glycero-3-phosphoinositol (DSPI), 1,2-dioleoyl-sn-glycero-3-phospho-(1′-myo-inositol) (DOPI), egg phosphatidylglycerol and soy phosphatidylglycerol.

In certain embodiments, the anionic lipid is POPG. In certain embodiments, the anionic lipid is DSPG. In certain embodiments, the anionic lipid is DPPG. In certain embodiments, the anionic lipid is DOPG. In certain embodiments, the anionic lipid is DOPG. In certain embodiments, the anionic lipid is DOPG. In certain embodiments, the anionic lipid is POPI. In certain embodiments, the anionic lipid is DOPG. In certain embodiments, the anionic lipid is DOPI. In certain embodiments, the anionic lipid is egg phosphatidylglycerol. In certain embodiments, the anionic lipid is soy phosphatidylglycerol.

In some embodiments, the first lipid comprises at least one unsaturated carbon-carbon bond. In certain embodiments, at least one first lipid is a phospholipid selected from the group consisting of 1,2-dioleoyl-sn-glycero-3-phosphoethanolamine-N-[4-(p-maleimidophenyl) butyramide] (MPB-PE), 1,2-diolcoyl-sn-glycero-3-phosphoethanolamine-maleimide (DOPE-Mal), 1,2-diolcoyl-sn-glycero-3-phosphoethanolamine-N-hydroxysuccinimide (DOPE-NHS), 1,2-dioleoyl-sn-glycero-3-phosphoethanolamine-azide (DOPE-azide), 1,2-dioleoyl-sn-glycero-3-phosphoethanolamine-dibenzocyclooctyne (DOPE-DBCO), and 1,2-dioleoyl-sn-glycero-3-phosphoethanolamine-trans-cyclooctene (DOPE-TCO). In some embodiments, the first lipid is DOPE-Mal. In certain embodiments, the first lipid is DOPE-NHS. In certain embodiments, the first lipid is DOPE-azide. In certain embodiments, the first lipid DOPE-DBCO. In certain embodiments, the first lipid is DOPE-TCO. In certain embodiments, the first lipid is MPB-PE.

In some embodiments, the liposome comprises at least two first lipids independently selected from the group consisting of MPB-PE, DOPE-Mal, DOPE-NHS, DOPE-azide, DOPE-DBCO, DOPE-TCO, and DSPC. In certain embodiments, the liposome comprises the first lipids, PB-PE and DOPE-Mal. In certain embodiments, the liposome comprises the first lipids, MPB-PE and DOPE-NHS. In certain embodiments, the liposome comprises the first lipids, MPB-PE and DOPE-azide. In certain embodiments, the liposome comprises the first lipids, MPB-PE and DOPE-DBCO. In certain embodiments, the liposome comprises the first lipids, MPB-PE and DOPE-TCO. In certain embodiments, the liposome comprises the first lipids, MPB-PE and DSPC. In certain embodiments, the liposome comprises the first lipids, DOPE-Mal and DOPE-azide. In certain embodiments, the liposome comprises the first lipids, DOPE-Mal and DOPE-NHS. In certain embodiments, the liposome comprises the first lipids, DOPE-Mal and DOPE-DBCO. In certain embodiments, the liposome comprises the first lipids, DOPE-Mal and DOPE-TCO. In certain embodiments, the liposome comprises the first lipids, DOPE-Mal and DSPC. In certain embodiments, the liposome comprises the first lipids, DOPE-NHS and DOPE-azide. In certain embodiments, the liposome comprises the first lipids, DOPE-NHS and DOPE-DBCO. In certain embodiments, the first lipids are DOPE-NHS and DOPE-TCO. In certain embodiments, the liposome comprises the first lipids, DOPE-NHS and DSPC. In certain embodiments, the liposome comprises the first lipids, DOPE-azide and DOPE-DBCO. In certain embodiments, the liposome comprises the first lipids, DOPE-azide and DOPE-TCO. In certain embodiments, the liposome comprises the first lipids, DOPE-azide and DSPC. In certain embodiments, the liposome comprises the first lipids, DOPE-DBCO and DOPE-TCO. In certain embodiments, the liposome comprises the first lipids, DOPE-DBCO and DSPC. In certain embodiments, the liposome comprises the first lipids, DOPE-TCO and DSPC.

In some embodiments, the liposome comprises at least three first lipids independently selected from the group consisting of MPB-PE, DOPE-Mal, DOPE-NHS, DOPE-azide, DOPE-DBCO, DOPE-TCO, and DSPC.

In certain embodiments, the liposome comprises 25-90% of a first lipid, 1-45% of a sterol, 3-30% of an anionic lipid, and 1-10% of a second lipid. In certain embodiments, the liposome comprises at least 25% of a first lipid. In certain embodiments, the liposome comprises at most 90% of a first lipid. In some embodiments, the liposome comprises 25-35% of a first lipid. In some embodiments, the liposome comprises 25-45% of a first lipid. In some embodiments, the liposome comprises 25-55% of a first lipid. In some embodiments, the liposome comprises 55-65% of a first lipid. In some embodiments, the liposome comprises 35-65% of a first lipid. In some embodiments, the liposome comprises 45-65% of a first lipid. In some embodiments, the liposome comprises 60-65% of a first lipid. In some embodiments, the liposome comprises 55-65% of a first lipid. In some embodiments, the liposome comprises 60-70% of a first lipid. In some embodiments, the liposome comprises 60-90% of a first lipid. In some embodiments, the liposome comprises 65-75% of a first lipid. In some embodiments, the liposome comprises 75-90% of a first lipid. In some embodiments, the liposome comprises 80-90% of a first lipid. In some embodiments, the liposome comprises 85-90% of a first lipid. In certain embodiments, the liposome does not comprise any lipid apart from the first lipid.

In certain embodiments, the liposome comprises at least 25% DSPC. In certain embodiments, the liposome comprises at most 90% DSPC. In some embodiments, the liposome comprises 25-35% DSPC. In some embodiments, the liposome comprises 25-45% DSPC. In some embodiments, the liposome comprises 25-55% DSPC. In some embodiments, the liposome comprises 55-65% DSPC. In some embodiments, the liposome comprises 35-65% DSPC. In some embodiments, the liposome comprises 45-65% DSPC. In some embodiments, the liposome comprises 60-65% DSPC. In some embodiments, the liposome comprises 55-65% DSPC. In some embodiments, the liposome comprises 60-70% DSPC. In some embodiments, the liposome comprises 60-90% DSPC. In some embodiments, the liposome comprises 65-75% DSPC. In some embodiments, the liposome comprises 75-90% DSPC. In some embodiments, the liposome comprises 80-90% DSPC. In some embodiments, the liposome comprises 85-90% DSPC. In certain embodiments, the liposome does not comprise a sterol.

In certain embodiments, the liposome comprises at least 1% of a sterol. In some embodiments, the liposome comprises at most 45% of a sterol. In some embodiments, the liposome comprises 0-15% of a sterol. In some embodiments, the liposome comprises 1-5% of a sterol. In some embodiments, the liposome comprises 5-10% of a sterol. In some embodiments, the liposome comprises 15-25% of a sterol. In some embodiments, the liposome comprises 15-35% of a sterol. In some embodiments, the liposome comprises 25-35% of a sterol. In some embodiments, the liposome comprises 25-45% of a sterol. In some embodiment, the liposome comprises 35-45% of a sterol. In some embodiments, the liposome comprises 20-25% of a sterol. In some embodiments, the liposome comprises 24-26% of a sterol. In some embodiments, the liposome comprises 20-30% of a sterol. In certain embodiments, the liposome does not comprise a sterol.

In certain embodiments, the liposome comprises at least 15% cholesterol. In some embodiments, the liposome comprises at most 45% cholesterol. In some embodiments, the liposome comprises 0-15% cholesterol. In some embodiments, the liposome comprises 1-5% cholesterol. In some embodiments, the liposome comprises 5-10% cholesterol. In some embodiments, the liposome comprises 15-25% cholesterol. In some embodiments, the liposome comprises 15-35% cholesterol. In some embodiments, the liposome comprises 25-35% cholesterol. In some embodiments, the liposome comprises 25-45% cholesterol. In some embodiment, the liposome comprises 35-45% cholesterol. In some embodiments, the liposome comprises 20-25% cholesterol. In some embodiments, the liposome comprises 24-26% cholesterol. In some embodiments, the liposome comprises 20-30% cholesterol.

In some embodiments, the liposome comprises at last 3% of an anionic lipid. In some embodiments, the liposome comprises at most 30% of an anionic lipid. In some embodiments, the liposome comprises 3-10% of an anionic lipid. In some embodiments, the liposome comprises 4-8% of an anionic lipid. In some embodiments, the liposome comprises 5-7% of an anionic lipid. In some embodiments, the liposome comprises 3-20% of an anionic lipid. In some embodiments, the liposome comprises 10-20% of an anionic lipid. In some embodiments, the liposome comprises 10-30% of an anionic lipid. In some embodiments, the liposome comprises 20-30% of an anionic lipid.

In some embodiments, the liposome comprises at last 3% POPG. In some embodiments, the liposome comprises at most 30% POPG. In some embodiments, the liposome comprises 3-10% POPG. In some embodiments, the liposome comprises 4-8% POPG. In some embodiments, the liposome comprises 5-7% POPG. In some embodiments, the liposome comprises 3-20% POPG. In some embodiments, the liposome comprises 10-20% POPG. In some embodiments, the liposome comprises 10-30% POPG. In some embodiments, the liposome comprises 20-30% POPG.

In some embodiments, the liposome comprises at least 1% of a phospholipid. In some embodiments, the liposome comprises at most 10% of a phospholipid. In some embodiments, the liposome comprises 1-5% of a phospholipid. In some embodiments, the liposome comprises 3-8% of a phospholipid. In some embodiments, the liposome comprises 4-6% of a phospholipid. In some embodiments, the liposome comprises 5-10% of a phospholipid. In some embodiments, the liposome me comprises 8-10% of a phospholipid.

In some embodiments, the liposome comprises at least 1% MPB-PE. In some embodiments, the liposome comprises at most 10% MPB-PE. In some embodiments, the liposome comprises 1-5% MPB-PE. In some embodiments, the liposome comprises 3-8% MPB-PE. In some embodiments, the liposome comprises 4-6% MPB-PE. In some embodiments, the liposome comprises 5-10% MPB-PE. In some embodiments, the liposome me comprises 8-10% MPB-PE.

In certain embodiments, the liposome comprises 25-90% DSPC, 0-45% cholesterol, 3-30% POPG, and 1-10% MPB-PE. In certain embodiments, the liposome comprises 35-75% DSPC, 10-30% cholesterol, 2-10% POPG, and 3-8% MPB-PE. In certain embodiments, the liposome comprises 60-70% DSPC, 20-30% cholesterol, 4-8% POPG, and 4-6% MPB-PE. In some embodiments, the liposome comprises 65% DSPC, 24% cholesterol, 6% POPG, and 5% MPB-PE.

In some embodiments, the first lipid further comprises a linker. In certain embodiments, the linker comprises at least one polyethylene glycol (PEG). In certain embodiments, the linker comprises at least two PEGS. In certain embodiments, the linker comprises at least three PEGs. In some embodiments, the linker is at least one amino acid. In some embodiments, the linker is polyglycine. In some embodiments, the linker is PMMA. In certain embodiments, the linker is PPG. In certain embodiments, the first lipid is covalently linked to the immunostimulatory agent by a linker.

In certain embodiments, the first lipid comprises a sterol. In certain embodiments, the first lipid comprises a cholesterol. In certain embodiments, the first lipid comprises a sterol further comprising a linker. In certain embodiments, the first lipid comprises cholesterol further comprising a linker.

In certain embodiments, the first lipid is covalently bonded to the immunostimulatory agent via maleimide-cysteine conjugation, N-hydroxysuccinimide easter-amine crosslinking, click chemistry, Staudinger ligation, Diels-alder cycloaddition, photoinducible ligation, oxime-hydrazone ligation, tetrazine ligation, palladium-ruthenium catalysis, disulfide formation, or Michael addition. In some embodiments, the first lipid is bonded to the immunostimulatory agent via an nucleophile-electrophile reaction. In certain embodiments, the first lipid is covalently bound to the immunostimulatory agent via maleimide-cysteine conjugation. In certain embodiments, the first lipid is covalently bonded to the immunostimulatory agent via N-hydroxysuccinimide caster-amine crosslinking. In certain embodiments, the first lipid is covalently bonded to the immunostimulatory agent via click chemistry. In certain embodiments, the first lipid is covalently bonded to the immunostimulatory agent via Staudinger ligation. In certain embodiments, the first lipid is covalently bonded to the immunostimulatory agent via Diels-alder cycloaddition. In certain embodiments, the first lipid is covalently bonded to the immunostimulatory agent via photoinducible ligation. In certain embodiments, the first lipid is covalently bonded to the immunostimulatory agent via oxime-hydrazone ligation. In certain embodiments, the first lipid is covalently bonded to the immunostimulatory agent via tetrazine ligation. In certain embodiments, the first lipid is covalently bonded to the immunostimulatory agent via palladium-ruthenium catalysis. In certain embodiments, the first lipid is covalently bonded to the immunostimulatory agent via disulfide formation. In certain embodiments, the first lipid is covalently bonded to the immunostimulatory agent via Michael addition. In certain embodiments, the first lipid is covalently bonded to the immunostimulatory agent via a linker.

In some embodiments, the polycation is selected from the group consisting of poly-L-arginine (PLR), poly-L-lysine (PLL), polyethylenimine (PEI), poly(diallyldimethylammonium chloride) (PDAC), poly(β-amino esters) (PBAE), and poly(allylamine hydrochloride) (PAH), or a combination or derivative thereof. In some embodiments, the polycation is poly-L-arginine (PLR). In some embodiments, the polycation is PLL. In some embodiments, the polycation is PEI. In certain embodiments, the polycation is PDAC. In some embodiments, the polycation is a poly β-amino ester. In some embodiments, the polycation is PAH. In certain embodiments, there are at least one polycations selected from the group consisting of PLR, PLL, PEI, PDAC, PBAE and PAH. In certain embodiments, the particle comprises least two polycations selected from the group consisting of PLR, PLL, PEI, PDAC, PBAE and PAH. In certain embodiments, the particle comprises the polycations, PLR and PLL. In certain embodiments, the particle comprises the polycations, PLR and PEI. In certain embodiments, the particle comprises the polycations, PLR and PDAC. In certain embodiments, the particle comprises the polycations, PLR and PBAE. In certain embodiments, the particle comprises the polycations, PLR and PAH. In certain embodiments, the particle comprises the polycations, PLL and PEI. In certain embodiments, the particle comprises the polycations, PLL and PDAC. In certain embodiments, the particle comprises the polycations, PLL and PBAE. In certain embodiments, the particle comprises the polycations, PLL and PAH. In certain embodiments, the particle comprises the polycations, PEI and PDAC. In certain embodiments, the particle comprises the polycations, PEI and PBAE. In certain embodiments, the particle comprises the polycations, PEI and PAH. In certain embodiments, the particle comprises the polycations, PDAC and PBAE. In certain embodiments, the particle comprises the polycations, PDAC and PAH. In certain embodiments, the particle comprises the polycations, c PBAE and PAH.

In certain embodiments, the polyanion is selected from the group consisting of poly-L-glutamic acid (PLE), poly-L-aspartic acid (PLD), poly-hyaluronic acid (HA), poly-acrylic acid (PAA), and poly(sodium 4-styrenesulfonate), or a combination or derivative thereof. In certain embodiments, the polyanion is selected from the group consisting of poly-L-glutamic acid (PLE), poly-acrylic acid (PAA), and poly(sodium 4-styrenesulfonate), or a combination or derivative thereof. In certain embodiments, the polyanion is PLE. In certain embodiments, the polyanion is PAA. In some embodiments, the polyanion is poly(sodium 4-styrenesulfonate). In some embodiments, there is at least one polyanion. In certain embodiments, the particle comprises at least two polyanions selected from the group consisting of PLE, PLD, HA, PAA, and poly(sodium 4-styrenesulfonate), or a combination or derivative thereof. In certain embodiments the particle comprises at least three polyanions selected from the group consisting of PLE, PLD, HA, PAA, and poly(sodium 4-styrenesulfonate), or a combination or derivative thereof. In certain embodiments, the particle comprises the polyanions, PLE and PLD. In some embodiments, the particle comprises the polyanions, PLE and HA. In some embodiments, the particle comprises the polyanions, PLE and PAA. In some embodiments, the particle comprises the polyanions, PLE and poly(sodium 4-styrenesulfonate). In some embodiments, the particle comprises the polyanions, PLD and poly(sodium 4-styrenesulfonate). In some embodiments, the particle comprises the polyanions, HA and poly(sodium 4-styrenesulfonate). In some embodiments, the particle comprises the polyanions, PAA and poly(sodium 4-styrenesulfonate). In some embodiments, the particle comprises the polyanions, PAA and PLD. In some embodiments, the particle comprises the polyanions, PAA and HA.

In certain embodiments, the immunostimulatory agent is selected from the group consisting of a cytokine, an antibody, an affibody, a nanobody, a protein, a peptide, and variants and combinations of the foregoing.

In certain embodiments, the immunostimulatory agent is a cytokine. In certain embodiments, the immunostimulatory agent is an antibody. In certain embodiments, the immunostimulatory agent is an affibody. In certain embodiments, the immunostimulatory agent is a nanobody. In certain embodiments, the immunostimulatory agent is a protein. In certain embodiments, the immunostimulatory agent is a peptide. In certain embodiments, the immunostimulatory agent is a small molecule.

In some embodiments the immunostimulatory agent is a cytokine or an antibody. In certain embodiments, the immunostimulatory agent is a cytokine.

In certain embodiments, the cytokine is selected from the group consisting of interleukin-12 (IL-12), interleukin-2 (IL-2), interferon-γ (IFN-γ), interferon-α (IFN-α), interferon-β (IFN-β), interleukin-15 (IL-15), interleukin-15 super agonist (IL-15SA), interleukin-18 (IL-18), tumor necrosis factor alpha (TNF-α), interleukin-10 (IL-10), interleukin-8 (IL-8), TNF-related apoptosis-inducing ligand (TRAIL), FMS-like tyrosine kinase 3 ligand (FLT3LG), and variants thereof. In certain embodiments, at least one cytokine is selected from the group consisting of IL-12, IL-2, IFN-γ, IFN-α, IFN-β, IL-15, IL-15SA, IL-18, TNF-α, IL-10, IL-8, TRAIL, FLT3L3, and variants thereof.

In some embodiments, the cytokine is IL-2 or a variant thereof. In some embodiments, the cytokine is IFN-γ or a variant thereof. In certain embodiments, the cytokine is IFN-α or a variant thereof. In certain embodiments, the cytokine is IFN-β, or a variant thereof. In certain embodiments, the cytokine is IL-15, or a variant thereof. In certain embodiments, the cytokine is IL-15SA, or a variant thereof. In certain embodiments, the cytokine is IL-18, or a variant thereof. In certain embodiments, the cytokine is TNF-α, or a variant thereof. In certain embodiments, the cytokine is IL-10, or a variant thereof. In certain embodiments, the cytokine is IL-8, or a variant thereof. In certain embodiments, the cytokine is TRAIL, or a variant thereof. In certain embodiments, the cytokine is FLT3L3 or a variant thereof.

In certain embodiments, the cytokine is single chain (sc) IL-12, or a variant thereof. In some embodiments, the cytokine is human scIL-12.

In some embodiments, at least one immunostimulatory agent is an antibody. In certain embodiments, the antibody is selected from the group consisting of anti-PD1 antibodies, anti-PDL1 antibodies, anti-CTLA4 antibodies, anti-TIM-3 antibodies, anti-LAG-3 antibodies, anti-NKG2A antibodies, anti-CD73 antibodies, anti-A2aR antibodies, anti-B7-H3 antibodies, anti-B7-H4 antibodies, and variants thereof. In certain embodiments, at least one antibody is selected from the group consisting of anti-PD1 antibodies, anti-PDL1 antibodies, anti-CTLA4 antibodies, anti-TIM-3 antibodies, anti-LAG-3 antibodies, anti-NKG2A antibodies, anti-CD73 antibodies, anti-A2aR antibodies, anti-B7-H3 antibodies, anti-B7-H4 antibodies, and variants thereof. In certain embodiments, at least two antibodies are independently selected from the group consisting of anti-PD1 antibodies, anti-PDL1 antibodies, anti-CTLA4 antibodies, anti-TIM-3 antibodies, anti-LAG-3 antibodies, anti-NKG2A antibodies, anti-CD73 antibodies, anti-A2aR antibodies, anti-B7-H3 antibodies, anti-B7-H4 antibodies, and variants thereof.

In certain embodiments, the antibody is an anti-PD1 antibody. In certain embodiments, the antibody is an anti-PDL1 antibody. In certain embodiments, the antibody is an anti-CTLA4 antibody. In certain embodiments, the antibody is an anti-TIM-3 antibody. In certain embodiments, the antibody is an anti-LAG-3 antibody. In certain embodiments, the antibody is an anti-NKG2A antibody. In certain embodiments, the antibody is an anti-CD73 antibody. In certain embodiments, the antibody is anti-A2aR antibody. In certain embodiments, the antibody is an anti-B7-H3 antibody. In certain embodiments, the antibody is an anti-B7-H4 antibody. In certain embodiments, the antibody is a variant of the described antibodies.

In some embodiments, the particle described herein further comprising an additional therapeutic agent.

In certain embodiments, the particle described herein comprises: the liposome, further comprising: the first lipid, MPB-PE, covalently bound to the immunostimulatory agent, IL-12; 65% DSPC, 24% cholesterol, and 6% POPG; and the polymer coating comprising a PLR layer that is non-covalently associated with the outer surface of the liposome; and a PLE layer that is non-covalently associated with the at least one polycation layer.

In certain embodiments, the polymer coating reduces endocytosis of the particle by a target cell. In some embodiments, the polymer coating reduces endocytosis of the immunostimulatory agent by a target cell. In some embodiments, the polymer coating facilitates association of the particle with a target cell.

In some embodiments, the target cell is a cancer cell. In some embodiments, the target cell is a healthy cell. In certain embodiments, the cancer cell is selected from the group consisting of an ovarian cancer cell, a colon cancer cell, breast cancer cell, head and neck cancer cell, lung cancer cell, and brain cancer cell. In certain embodiments, the cancer cell is an ovarian cancer cell. In certain embodiments, the cancer cell is a colon cancer cell. In certain embodiments, the cancer cell is a head and neck cancer cell. In certain embodiments, the cancer cell is a brain cancer cell. In certain embodiments, the cancer cell is a lung cancer cell.

In one aspect, provided herein a pharmaceutical composition comprising a plurality of particles of the define composition and an a pharmaceutically acceptable excipient. In certain embodiments, the pharmaceutical composition further comprises an additional pharmaceutical agent. In certain embodiments, the additional pharmaceutical agent is selected from the group consisting of a chemotherapeutic, targeted therapy, gene therapy, immune therapy, and hormone therapy. In certain embodiments, the additional pharmaceutical is chemotherapeutic. In certain embodiments, the additional pharmaceutical is a targeted therapy. In certain embodiments, the additional pharmaceutical is a gene therapy. In certain embodiments, the additional pharmaceutical is a hormone therapy. In certain embodiments, the additional pharmaceutical agent is an immune therapy. In some embodiments, the immune therapy is immune checkpoint therapy.

In some embodiments, the immune checkpoint therapy is selected from the group consisting of anti-PD1 immune checkpoint therapy, anti-PDL1 immune checkpoint therapy, anti-CTLA4 immune checkpoint therapy, anti-TIM-3 immune checkpoint therapy, anti-LAG-3 immune checkpoint therapy, anti-NKG2A immune checkpoint therapy, anti-CD73 immune checkpoint therapy, anti-A2aR immune checkpoint therapy, anti-B7-H3 immune checkpoint therapy, and anti-B7-H4 immune checkpoint therapy, or a combination thereof.

In some embodiments, the immune checkpoint therapy is anti-PDI immune checkpoint therapy, anti-PDL1 immune checkpoint therapy, or anti-CTLA4 immune checkpoint therapy, or a combination thereof.

In some embodiment, the immune checkpoint therapy is anti-CTLA-4 immune checkpoint therapy. In some embodiments, the anti-CTLA-4 immune checkpoint therapy is selected from the group consisting of ipilimumab and tremelimumab. In some embodiments, the anti-CTLA-4 immune checkpoint therapy is ipilimumab. In some embodiments, the anti-CTLA-4 immune checkpoint therapy is tremelimumab.

In certain embodiments, the immune checkpoint therapy is anti-PDI immune checkpoint therapy. In certain embodiments, the anti-PDI immune checkpoint therapy is selected from the group consisting of pembrolizumab, nivolumab, and cemiplimab. In certain embodiments, the anti-PD1 immune checkpoint therapy is pembrolizumab. In certain embodiments, the anti-PD1 immune checkpoint therapy is nivolumab. In certain embodiments, the anti-PD1 immune checkpoint therapy is cemiplimab.

In certain embodiments, the immune checkpoint therapy is anti-PDL1 immune checkpoint therapy. In certain embodiments, the anti-PDL1 immune checkpoint therapy is selected from the group consisting of atezolizumab, avelumab, and durvalumab. In certain embodiments, the anti-PDL1 immune checkpoint therapy is atezolizumab. In certain embodiments, the anti-PDL1 immune checkpoint therapy is avelumab. In certain embodiments, the anti-PDL1 immune checkpoint therapy is durvalumab.

In one aspect, provided in the present disclosure is a particle or a pharmaceutical composition described herein, for use in treating a proliferative disease or immune disorder in a subject.

In another aspect, the present disclosure provides the use of a particle or a pharmaceutical composition described herein, for the manufacture of a medicament for treating proliferative disease or immune disorder in a subject.

In one aspect, provided herein a method of delivering an immunostimulatory agent to a target cell, wherein the method comprises contacting the target cell with a particle described herein.

In another aspect of the present disclosure, provided herein a method of recruiting immune cells to a target cell, wherein the method comprises contacting the target cell with a particle defined herein. In certain embodiments, the immunostimulatory agent is released from the particle. In some embodiments, the immunostimulatory agent associates with the cell membrane of the target cell. In certain embodiment, wherein the target cell is present in a subject or a biological sample, the first lipid undergoes lipid exchange with a biological component of the subject or of the biological sample. In certain embodiments, the biological component is a serum component. In some embodiments, the biological component is a tumor-related component. In certain embodiments, the biological component is a tumor cell membrane. In some embodiments, the subject is a human. In certain embodiments, the target cell is ex vivo. In certain embodiments, the target cell is a cancer cell.

In one aspect, provided in the present disclosure a method of treating a disease in a subject in need thereof, the method comprising administering to the subject an effective amount of a particle defined herein. In some embodiments, further comprising administering to the subject an additional pharmaceutical agent. In certain embodiments, the disease is a proliferative disease or an immune disorder. In certain embodiments, the disease is an immune disorder. In certain embodiments, the disease is an autoimmune disease. In certain embodiments, the disease is an inflammatory disorder. In certain embodiments, the disease is a proliferative disease. In some embodiments, the disease is cancer. In certain embodiments, the cancer is selected from the group consisting of colon cancer, ovarian cancer, breast cancer, head and neck cancer, lung cancer, and brain cancer. In some embodiments, the cancer is colon cancer. In some embodiments, the cancer is breast cancer. In some embodiments, the cancer is head and neck cancer. In some embodiments, the cancer is lung cancer. In some embodiments, the cancer is brain cancer. In some embodiments, the cancer is ovarian cancer.

In certain embodiments, the particle is administered via intravenous injection, intraperitoneal injection, intratumoral injection, oral intake, or inhalation. In certain embodiments, the particle is administered via intravenous injection. In certain embodiments, the particle is administered via oral intake. In certain embodiments, the particle is administered via inhalation. In certain embodiments, the particle is administered via intraperitoneal injection. In certain embodiments, the particle is administered via intratumoral injection.

In one aspect of the present disclosure, provided herein a kit comprising: a particle or a pharmaceutical composition described herein; and instructions for using the particle or the pharmaceutical composition.

### EXAMPLES

### Example 1: Biophysical Properties of Layer-by-Layer Nanoparticles (LbL-NPs) Surface Conjugated with IL-12 Via Nickel-Histidine Interaction Vs. Covalent Bonding

Materials: 1,2-distearoyl-sn-glycero-3-phosphocholine (DSPC), Cholesterol, 1-palmitoyl-2-oleoyl-sn-glycero-3-phospho-(1′-rac-glycerol) (sodium salt) (POPG), 1,2-diolcoyl-sn-glycero-3-phosphoethanolamine-N-dibenzocyclooctyl (DOPE-DBCO), and cholesterol were purchased from Avanti Polar Lipids. Poly-L-arginine (PLR) with a molecular weight (MW) of 9.6 kDa and poly-L-glutamic acid (PLE) with a MW of 15 kDa were purchased from Alamanda Polymers. BDP TMR azide (Lumiprobe) and BDP 630/650 azide (Lumiprobe) were conjugated to DOPE-DBCO in chloroform to generate DOPE-TMR and DOPE-630/650. Successful conjugation was validated via thin-layer chromatography which indicated <1% free dye.

Cell Culture: OV2944-HM-1 cells were acquired through Riken BRC and were cultured in α-MEM while MC38 were cultured in DMEM. Cell media was also supplemented with 10% FBS and penicillin/streptomycin with cells incubated in a 5% CO2 humidified atmosphere at 37° C. All cell lines were murine pathogen tested and confirmed mycoplasma negative by Lonza MycoAlert™ Mycoplasma Detection Kit.

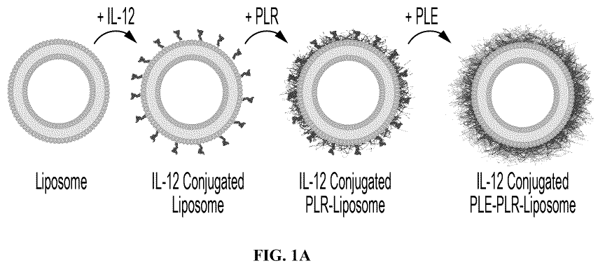

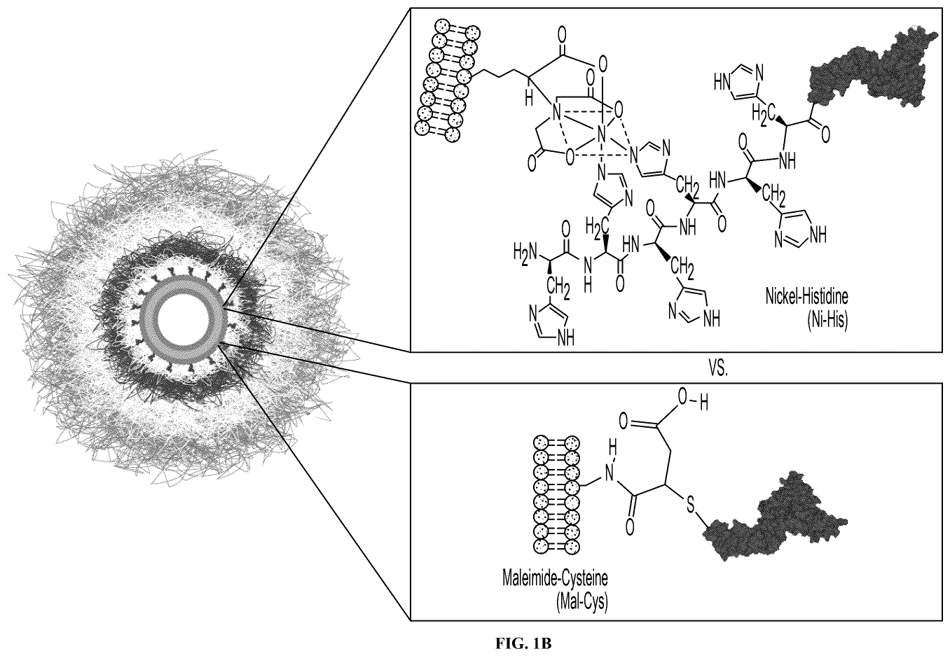

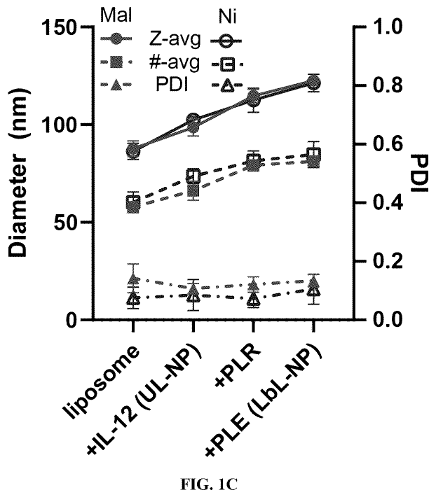

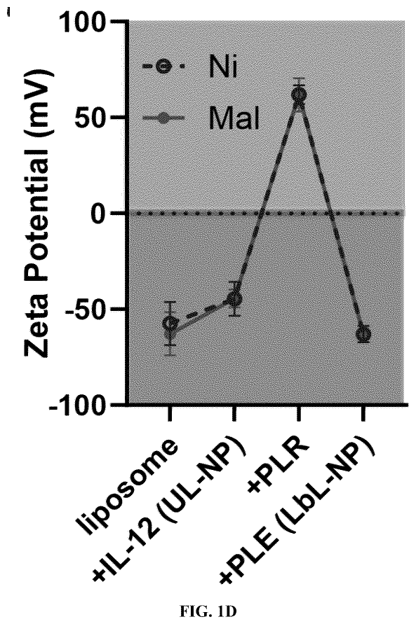

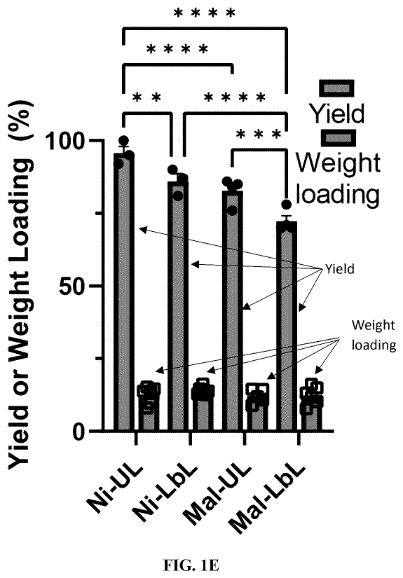

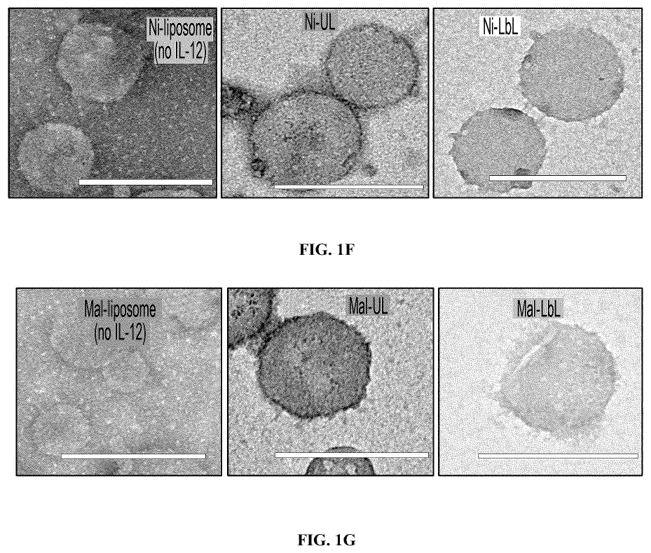

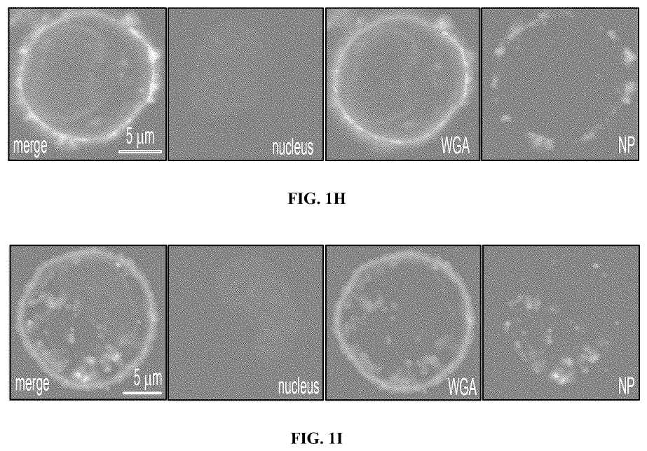

The overall design of ovarian cancer cell-targeting LbL-NPs is shown in FIGS. 1A-1B. An immunostimulatory payload (here, the potent cytokine IL-12) is linked to the surface of a liposomal core particle, followed by sequential LbL deposition of a layer of positively-charged polyarginine (PLR) and a layer of negatively charged poly-L-glutamate (PLE).

Liposome synthesis: Lipid solution composed of 65% DSPC, 24% Cholesterol, 6% POPG and 5% of either (Ni) NTA-DGS or MPB-PE were made in chloroform then formed into a thin film using a rotovap. Films were allowed to further dry overnight in a desiccator. Films were hydrated at 0.5-1 mg/mL using deionized water and sonicated for 3 minutes at 65° C. then extruded (Avestin Liposofast-50) at 65° C. once against a 100 nm membrane then thrice against 50 nm membranes. Extruded liposomes were added to an ice bath then scIL-12 was added to the lipid solution at 0.16 weight equivalents (wt.eq.) for Ni (NTA)-DGS liposomes at 0.5 mg/mL.

The solution pH of MPB-PE liposome was adjusted to pH 5 prior to hydration then adjusted to pH 7.0 with 10 mM HEPES prior to addition of scIL-12 containing a terminal cysteine residue at 0.17 wt.eq. with liposomes at 0.33 mg/mL. After overnight incubation with IL-12 at 4° C., Ni-UL liposomes were purified via tangential flow filtration on a 100 kDa mPES membranes (Repligen) against 6 diafiltration volumes of deionized water. For Mal-UL samples, any remaining maleimides were quenched with 100-fold molar excess of L-cysteine (Sigma) for 1.5 hours on ice. For fluorescence labeling of liposomes, 0.2 mol % of DSPC content was replaced by either DOPE-TMR or DOPE-630/650. IL-12 concentration was measured via enzyme-linked immunoassay (ELISA) (Peprotech) and lipid content was quantified via the Stewart Assay.

Layer-by-Layer (LbL) deposition: Assembly of polyelectrolyte layers was performed by adding unlayered particles to a solution with 0.3-0.4 wt.eq. of PLR relative to lipid in a glass vial under sonication and allowed to equilibrate on ice for at least 30 minutes. Excess PLR polymer was purified by TFF through a 100 kDa mPES membrane (Repligen) pre-treated with 10 mg/mL solution of free PLR. For the terminal PLE layer, purified particle coated with PLR were added to solution with PLE in a glass vial under sonication at 1 wt.eq. of polymer to lipid. Particles coated with both PLR and PLE were the purified by TFF on a separate 100 kDa mPES membrane (Repligen) to remove any excess PLE.

LbL-NPs where a single chain IL-12 molecule was anchored to the liposomal core by a non-covalent nickel: polyhistidine (Ni) interaction were tested, but were shown to be poorly stable in the presence of serum components. 12.13 The stability and efficacy of particles were compared where IL-12 was anchored via Ni-histag vs. a covalent thioether bond formed between a maleimide (Mal)-headgroup lipid incorporated in the lipid bilayer and a free cysteine introduced at the C-terminus of IL-12 (FIG. 1B). To prevent thiosuccinimide thiol-exchange or retro-Michael addition reaction, a lipid head group containing N-aryl maleimide was employed to favor thiosuccinimide hydrolysis into stable succinamic acid thioethers.¹⁸ Stable linkage to IL-12 was also promoted by using 300-fold molar excess of L-cysteine to quench unreacted maleimides and displace potential intermediate IL-12: thiosuccinimide groups via thiol exchange. IL-12-conjugated NPs were synthesized with both linker chemistries in either unlayered (UL) or PLR/PLE-layered (LbL) formats. These NPs had similar sizes and surface charges based on dynamic light scattering (DLS) (FIG. 1C) and zeta potential measurements (FIG. 1D), respectively.

Characterization of particle preparations: Dynamic light scattering (DLS) and zeta potential measurements were made on a Zetasizer Nano ZSP (Malvern). Nanoparticle micrographs were acquired using Transmission Electron Microscopy (TEM) on a JEOL 2100F microscope (200 kV) with a magnification range of 10,000-60,000×.

The Ni and Mal conjugation reactions were optimized to ensure high yields (>70%) and similar loadings of IL-12 (˜10-13 wt %) (FIG. 1E). IL-12 conjugation to the surface of liposomes were validated via negative-stain (NS) transmission electron micrography (TEM) where it was found that when stained with phosphotungstic acid, unlayered (UL) NPs without IL-12 presented the typical low contrast micrographs of liposomes. When conjugated with IL-12, however, a dark rim around the liposomes was observed which became diffuse after LbL deposition, indicating successful IL-12 conjugation and LbL coating (FIGS. 1F-1G). Mal-LbL NPs were incubated with OV2944-HM1 (hereafter HM-1), a highly metastatic OC cell line¹⁹, then analyzed NP subcellular localization via confocal imaging. For the selected LbL film chemistry (PLR/PLE), Mal-LbL NPs were primarily located on the cell membrane four hours after NP dosing and these NPs were found to have IL-12 by staining cells with an anti-IL-12 antibody (Ab) (FIG. 1H), demonstrating that maleimide conjugation of IL-12 did not interfere with the cancer cell membrane binding properties of PLR/PLE LbL-NPs. On the other hand, unlayered Mal NPs (Mal-UL), were endocytosed (FIG. 1I).

### Example 2: Stability of IL-12 Linkage to NPs and of Polyelectrolyte Film on LbL-NPs

To explore the effect of different conjugation chemistries on the in vitro IL-12 release kinetics from the NPs in either UL or LbL formats, IL-12 NPs were generated with fluorescently-tagged IL-12 and incubated in 10% or 100% fetal bovine serum (FBS) at 37° C. Samples from the incubated NPs were taken at various timepoints and were processed with a centrifugal filter to separate NPs from free IL-12. To avoid inducing IL-12 release during centrifugation, separation at low centrifugation speed (10 rcf) on a high molecular weight cut-off membrane (300 kDa) was performed.

In vitro cellular association: The day before dosing, HM-1 cells were plated on a tissue-culture 96-well plate at a density of 50 k cells per well. The next day, wells were dosed with NPs to 0.05 mg/mL and left for the target incubation time (4 hours or 24 hours). For analysis of association, the supernatant was removed from the well and diluted 10× with DMSO. Cells were then washed three times with PBS then dissolved with DMSO. Fluorescence of NPs associated with cells was then normalized to supernatant fluorescence. The relative fluorescence of each formulation was then compared to an unlayered liposome control containing the same fluorophore. For confocal imaging, 8-well chambered coverglass (Nunc Lab-Tek II, Thermo Scientific) were treated with rat tail collagen type I (Sigma-Aldrich) per manufacturer's instructions. HM-1 cells were plated onto wells at a density of 10 k/well and left to adhere overnight prior to NP treatment. After the desired incubation time with NPs, cells were washed 3× with PBS. After washing, cells were fixed in 4% paraformaldehyde for 10 minutes then washed (3× with PBS) and stained with wheat germ agglutinin (WGA) conjugated to Alexa Fluor488 (Invitrogen) and hoechst 33342 (Thermo Scientific) following manufacturer instructions. Images were analyzed using ImageJ. Slides were imaged on an Olympus FV1200 Laser Scanning Confocal Microscope. FRET studies were performed on a Leica SP8 Spectral Confocal Microscope.

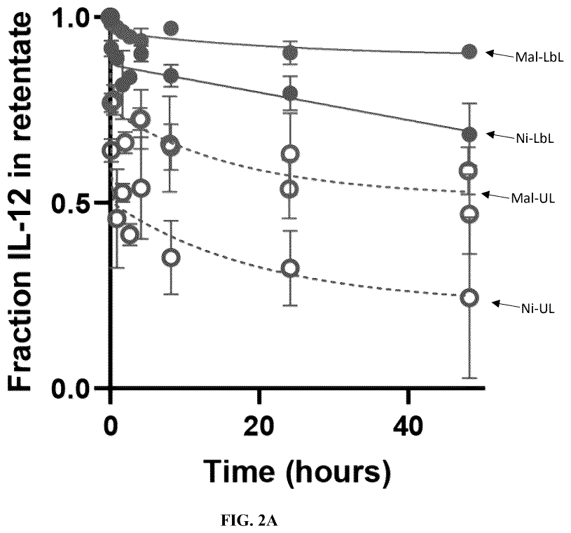

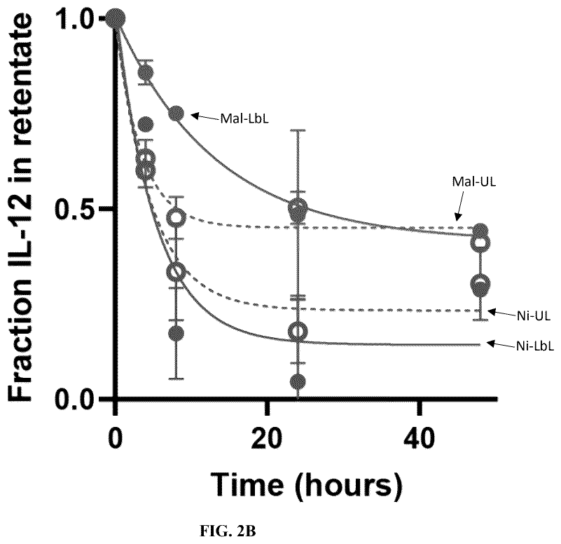

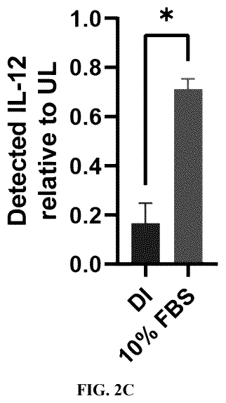

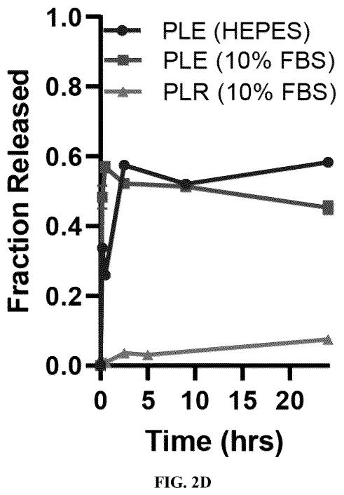

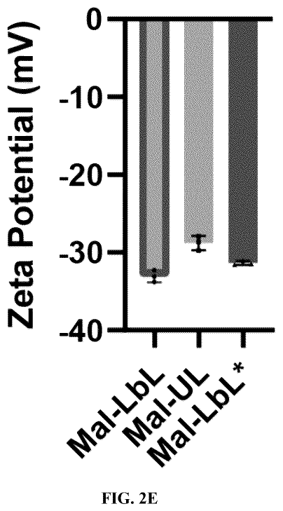

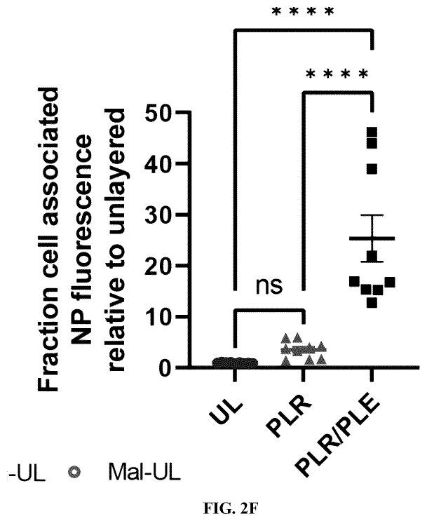

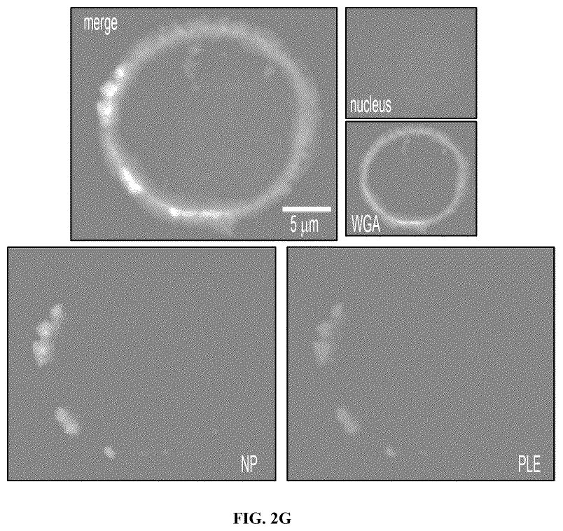

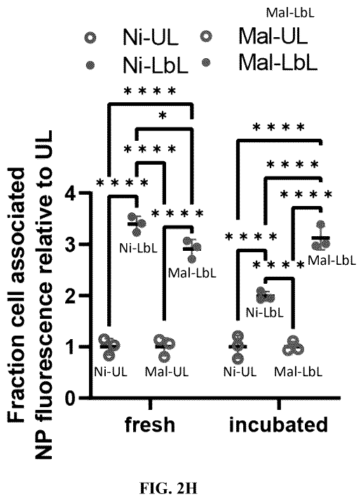

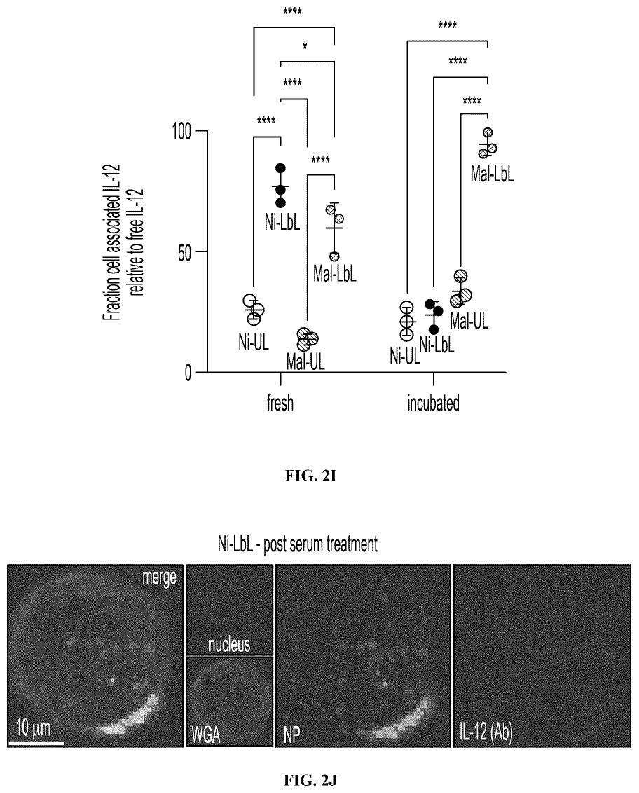

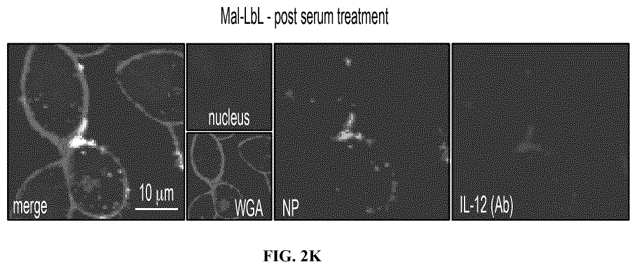

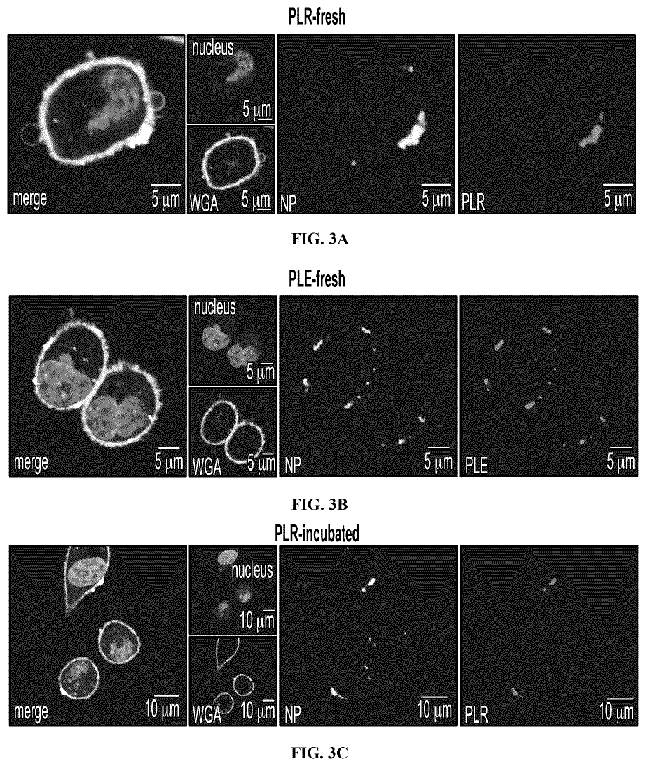

In 10% FBS, the data was well described by a two-phase decay model (FIG. 2A) where Ni-UL exhibited the fastest IL-12 release. Coating with polyelectrolytes increased IL-12 retention in Ni-LbL. Mal-UL NPs also released IL-12, indicating IL-12 detachment from the NPs even with a covalent lipid attachment. Mal-LbL exhibited the greatest IL-12 retention with an almost fivefold increase in half-lives. When exposed to 100% FBS (FIG. 2B), a simple one-phase decay model was sufficient to describe the data. Ni-UL, Ni-LbL and Mal-UL all presented similar half-lives of 2-3 hours with Mal-UL plateauing at a higher concentration of IL-12. Mal-LbL again exhibited the greatest retention of IL-12 with NPs with a half-life of 9 hours and plateauing to a similar range of Mal-UL. Unlike Ni particles which released free IL-12, the non-zero plateau of Mal NPs was indicative of samples approaching equilibrium level of IL-12 release. These data demonstrate the increased stability of IL-12 association with particles for Mal-LbL particles.

The effect and stability of the polyelectrolyte film on covalently conjugated NPs (Mal-LbL) was explored. Mal-LbL NPs were captured on microtiter plates in varying buffers and assessing the ability of an IL-12-specific monoclonal antibody to bind to the particles via enzyme-linked immunosorbent assay (ELISA). While LbL coating significantly reduced the IL-12 accessibility in deionized water, when placed in 10% FBS, the majority of IL-12 was available for binding to an ELISA plate (FIG. 2C). These results indicate that the polyelectrolyte films underwent rearrangements in physiological buffers, exposing the IL-12 from the NP surface.

Release of polymers in the LbL coating during these rearrangements was next explored. Mal-LbL NPs were generated with either PLR or PLE fluorescently tagged and measured their release kinetics from particles incubated in different buffer conditions. For PLE release, the low-speed spin filter centrifugation assay was performed to quantify free polymers released from NPs, similar to measurements of IL-12 release. As PLR was found to adhere to the spin filter membranes, instead free PLR were quantified in the supernatant following pelleting of the NPs by high-speed centrifugation. It was found a marked release of PLE when LbL-NP was exposed to either HEPES or 10% FBS, whereas PLR remained stably associated with the NP surfaces (FIG. 2D). However, enough PLE remained on the LbL-NPs to maintain an overall net negative charge (FIG. 2E). Further, when HM-1 cells were dosed with NP then had their cell-associated NP fluorescence measured in a plate reader, PLR/PLE LbL-NPs induce a significant increase in OC cell association compared to PLR only LbL-NPs (FIG. 2F), validating the requirement of PLE layering for optimal OC cell association. To validate retention of PLE after the initial burst release, it was observed via confocal microscopy that LbL-NPs treated with FBS still bound to cancer cell membranes and there was a clear colocalization of both PLE and PLR with the NPs after serum incubation (FIG. 2G, FIGS. 3A-3C).

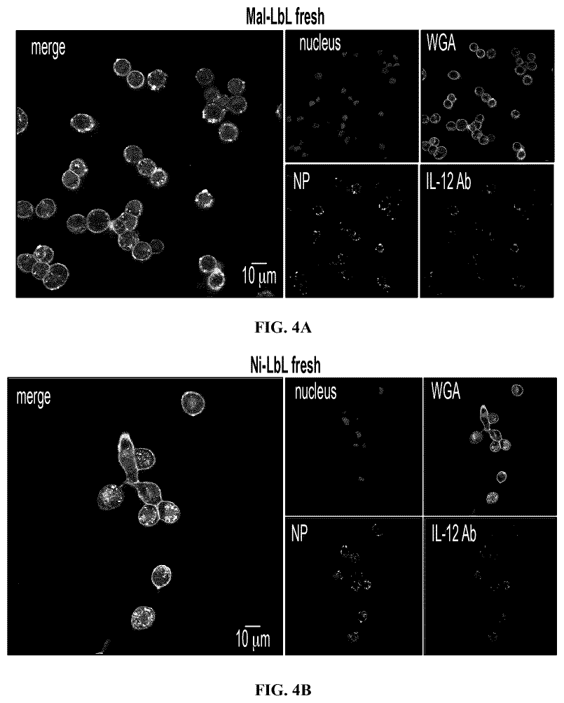

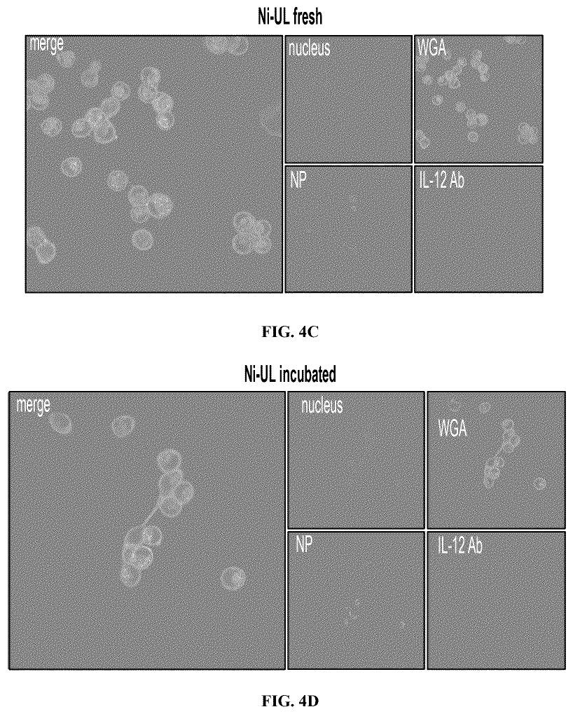

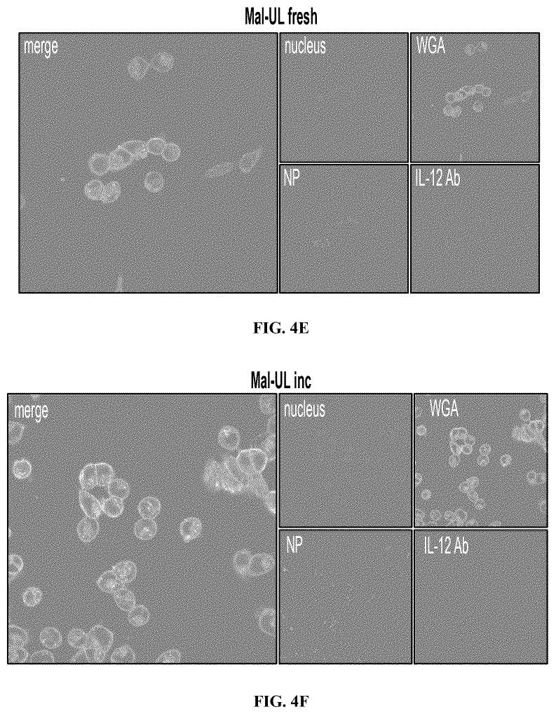

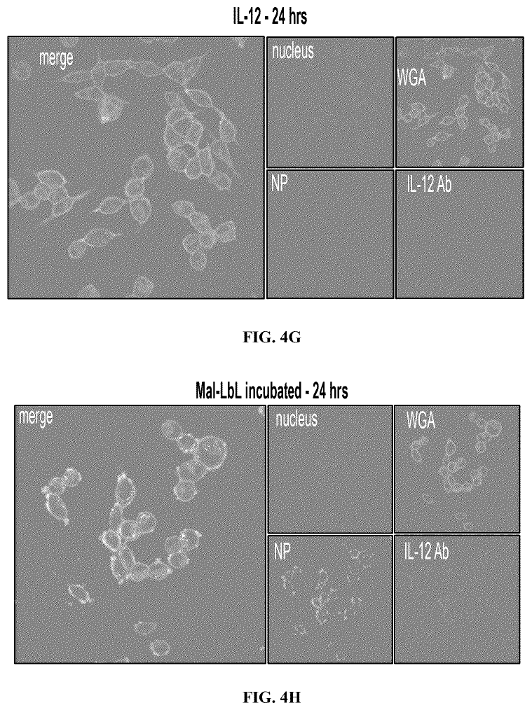

Given the increased IL-12 retention with Mal-LbL NPs and the strong LbL film stability, the ability of LbL-NPs to increase IL-12 association with OC and the impact of serum incubation of the NPs was explored next. NPs were dosed with fluorescently labeled lipids and IL-12 before (fresh) and after incubating (incubated) the NPs in 100% serum for 48 hours to HM-1 cells then measured the cell associated NP fluorescence in a plate reader. LbL-NPs in either the Mal-LbL or Ni-LbL formats had increased OC cell association, which was maintained after serum treatment, indicating that the polyelectrolyte film was still present on the NP after serum treatment (FIG. 2H). However, only Mal-LbL was able to maintain increased association of IL-12 with OC cell after serum treatment (FIG. 2I) which agreed with the IL-12 release studies (FIG. 2B). While fresh (non serum-exposed) Mal-LbL and Ni-LbL particles were confirmed to deliver IL-12 to OC cell membranes when analyzed via confocal imaging (FIGS. 4A-4B), unlayered Ni particles and serum-treated Ni-LbL NPs exhibited cell-membrane associated NPs that were devoid of IL-12 (FIG. 2J, FIGS. 4C-4D 4G). By contrast, Mal-LbL NPs retained high levels of associated IL-12 after serum incubation (FIG. 2K), which was retained on the cancer cell surfaces for at least 24 hours (FIG. 4H), demonstrating that Ni-LbL NPs have greater IL-12 release in the presence of serum compared to Mal-LbL in suspension. These data shows that serum incubation does not affect properties of Mal-LbL-NPs.

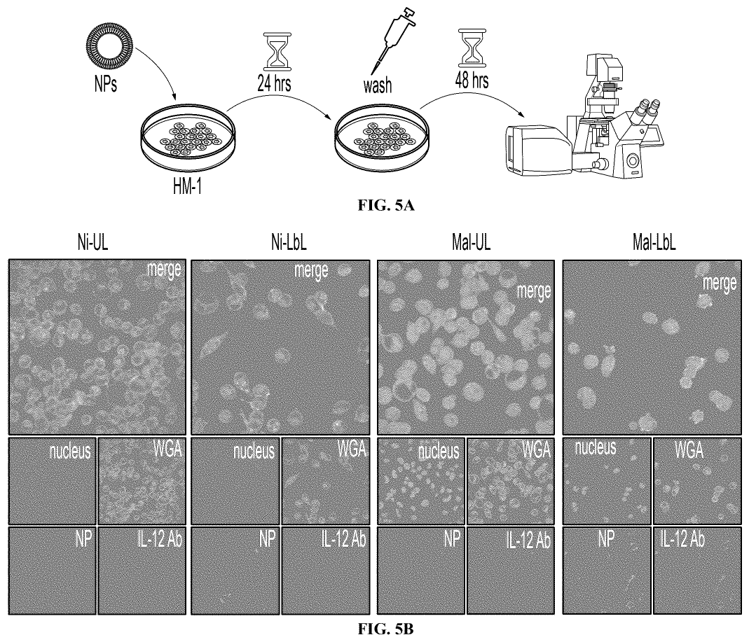

The ability of surface-bound Ni-LbL NPs to release IL-12 was next explored. HM-1 cells were dosed with IL-12 NPs for 24 hours to attach NPs to the cell surface, then washed the cells and waited for 48 hours before performing confocal imaging. While it was possible to observe surface-bound LbL-NPs 48 hours later, in the case of Ni-LbL, these were devoid of IL-12, but IL-12 was still present in surface-bound Mal-LbL NPs (FIG. 5A-B). These data shows that surface-bound Mal-LbL NPs retain IL-12 on the surface of cancer cells for over 48 hours whereas Ni-LbL releases IL-12.

### Example 3: Covalent Attachment of IL-12 to Phospholipids Enables IL-12 Delivery Via Lipid Exchange

Given the observation of IL-12 release from Mal NPs (FIGS. 2A-2B), it was theorized that this could be due to lipid detachment from the NPs resulting in an IL-12 amphiphile. Thus, it was assessed if detached fluorescently labeled lipids could be observed in the same centrifugal filter separation assay used for IL-12 release.

Lipid stability via lipid-lipid Förster resonance energy transfer (FRET) and donor dequenching: Lipid-lipid FRET was evaluated by generating liposomes with 0.5 mol % of both DOPE-TMR and DOPE-630/650 for unsaturated formulations and DPPC-TMR and DPPC-630 for saturated liposomes. FRET was quantified via the corrected FRET efficiency (FRETN) described previously.³⁸ Donor dequenching was also measured based on the corrected donor fluorescence.

IL-12 and lipid release assay: NPs with fluorescently tagged lipids and/or fluorescently tagged IL-12 were incubated in 10% or 100% fetal bovine serum (FBS) supplemented with 1× pen strep. For 10% FBS, FBS was diluted into a physiological buffer composed of 15 mM HEPES (pH 7.2) and 150 mM sodium chloride. Samples were kept at in a shaker at 37° C. and at set incubation periods, aliquots were taken from the incubation sample and placed on a 300 kDa Vivaspin 500 centrifugal filter (Sartorius). Filter were spun at 20 g for 20-30 minutes to obtain a permeate fraction devoid of NPs and its fluorescence was compared to the initial sample fluorescence to determine the total amount of IL-12 or lipid permeating the membrane. To account for partial IL-12 retention through the membrane, free IL-12 was also analyzed and used as a normalization factor.

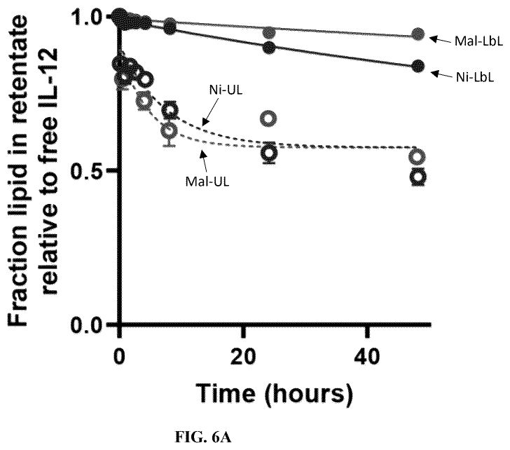

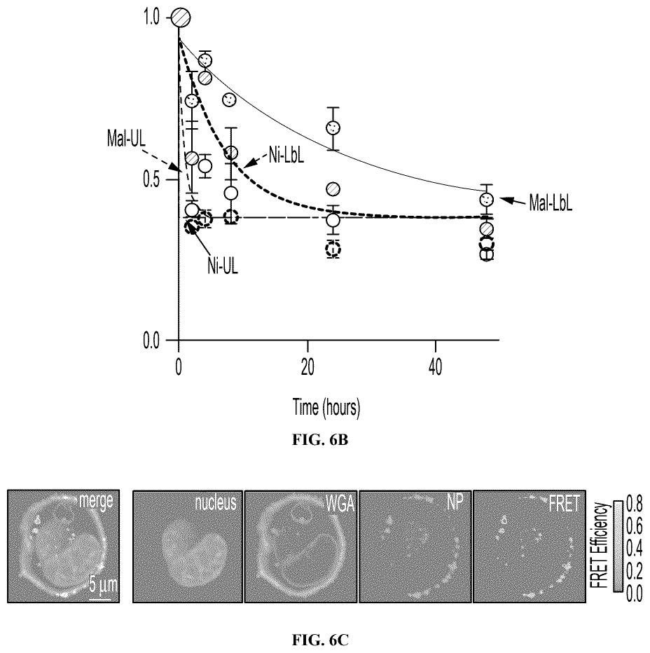

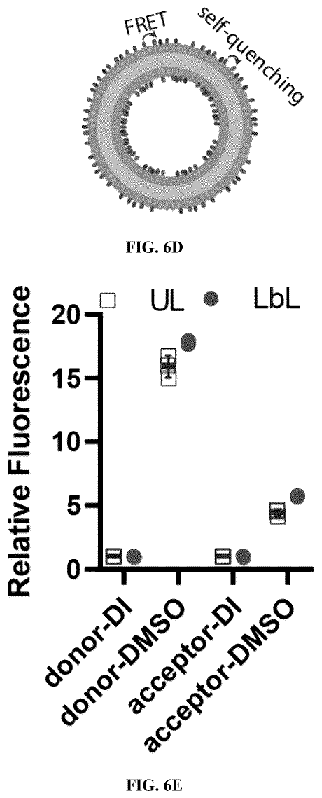

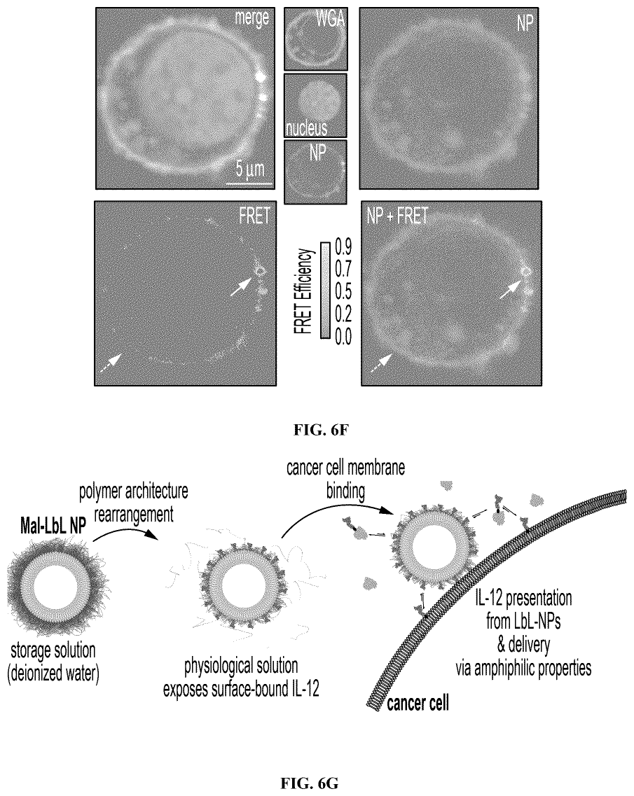

In 10% FBS (FIG. 6A), the data fit well to a one-phase decay model with a shared non-zero plateau. UL NPs had one order of magnitude faster half-lives compared to LbL-NPs with Mal-LbL exhibiting the largest half-life. In 100% FBS (FIG. 6B), the same model described the data well and the while all groups had significantly shorter half lives compared to 10% FBS incubation, LbL coating increased the calculated half-life. These results indicated that the liposomal NPs were releasing lipids and that the LbL film could protect them from serum components capable of mediating lipid release. Further, the presence of a non-zero plateaus was indicative of the NP reaching an equilibrium with the serum components. These plateaus were similarly observed in the IL-12 release profiles from Mal NPs (FIGS. 2A-2B), suggesting that IL-12 released as a lipid conjugate.

To assess liposome stability in the LbL-NP formulations, cell-surface associated fluorescence from NPs or free lipids was investigated. HM-1 cells were dosed with Mal-LbL NPs containing lipid-bound FRET pairs and used acceptor photobleaching FRET to evaluate if the NPs were intact or dissociated via confocal microscopy. It was found that the LbL-NPs bound to the cancer cell surface exhibited a high FRET efficiency, indicating that primarily intact NPs associated with the cancer cell membranes (FIG. 6C). To explore if local lipid release could mediate lipids to insert into the cancer cell membrane like previously reported amphiphile micelles²⁰, lipid-lipid FRET LbL-NPs were generated which contained high levels of dye to enable both FRET-induced donor quenching as well as donor self-quenching (FIG. 6D). With this approach, LbL-NPs could be generated in which the donor fluorescence was highly quenched in a NP form but could increase its fluorescence once dissociated from the NP (FIG. 6E). Indeed, when these LbL-NPs were tested via confocal microscopy, diffuse donor signal on the cell membrane could be readily observed. Furthermore, while surface bound NP depots showed high FRET efficiency, dyed lipids dispersed in the cell membrane did not, indicating disassembly of lipids from the NPs (FIG. 6F). Based on these observations, a model for targeting IL-12 to cancer cells via LbL-NPs in which the LbL film undergoes rapid structural changes and partial PLE release was developed. The polyelectrolyte film rearrangements reveal both IL-12 and lipids to interact with the surroundings while maintaining the targeting properties of the LbL coating. Exposure of the underlying core of bilayer LbL-NPs validates prior work in which the NP core appears accessible to interact with cells.^{21,22} For Mal-IL-12 NPs these interactions included directly engaging with IL-12 cell receptors in NP form or undergoing lipid-lipid exchange with serum proteins or, to a lower extent, cell-membrane lipids (FIG. 6G). These data shows that lipid release enables amphiphile-like properties to IL-12 in Mal-LbL NPs.

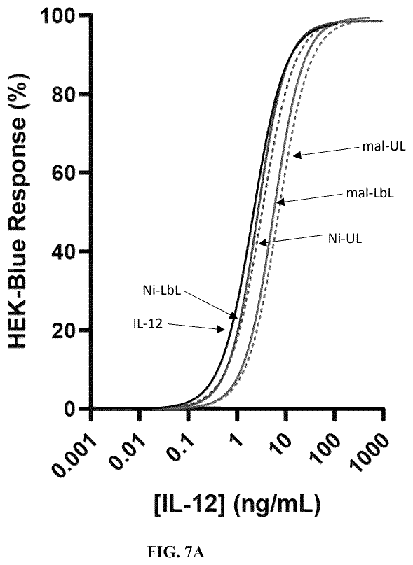

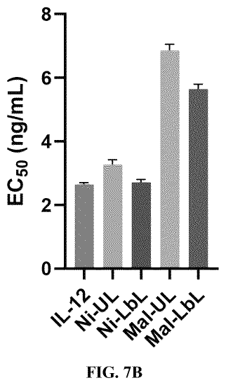

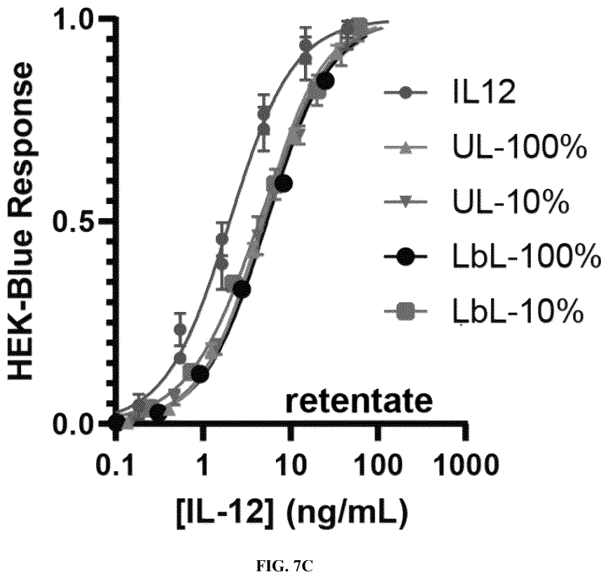

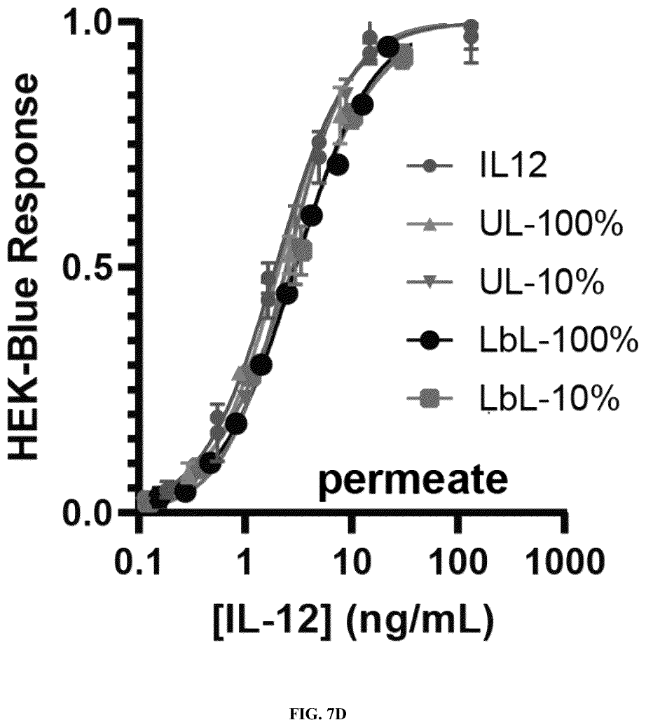

To better understand the impacts of the covalent lipid attachment, it was investigated whether the conjugation chemistry could alter the apparent IL-12 bioavailability by testing the Mal and Ni NPs in an IL-12 reporter cell line (HEK-Blue IL-12). IL-12 bioactivity was retained in either Ni or Mal NP constructs (FIGS. 7A-7B). Further, having exposed IL-12 on the LbL-NPs (FIG. 2C), yielded no major effect of LbL coating on the cell line reporter response (FIGS. 7A-7B). After incubating Mal NPs in 10% or 100% FBS for 48 hours, the IL-12 recovered in the retentate (NP rich) and in the permeate (released IL-12) post centrifugation continued to show bioactivity similar to free IL-12 (FIGS. 7C-7D). These data shows Mal conjugation of IL-12 to NPs alters apparent IL-12 bioactivity that is recoverable with IL-12 release.

### Example 4: Layer-by-Layer Nanoparticles Target Metastatic OC Tissue and Covalent IL-12 Conjugation Enables Prolonged Cytokine Retention in Tumor Tissue

It was next decided to investigate whether the varied conjugation chemistries would impact IL-12 trafficking in vivo by performing pharmacokinetics and biodistribution analysis in mice bearing intraperitoneally disseminated HM-1 ovarian tumors.

Mice: B6C3F1 mice were purchased from Jackson Laboratories. Female mice were used between 8-12 weeks of age unless otherwise noted. All animal work was conducted under the approval of the Massachusetts Institute of Technology Division of Comparative Medicine in accordance with federal, state, and local guidelines.

Fluorescently labeling of IL-12: IL-12 was labeled with indocyanine green (ICG) tetrafluorophenyl (TFP) ester (AAT Bioquest) by solubilizing the dye in dimethyl sulfoxide at 1 mg/mL and adding it to IL-12 at 3 mg/mL in phosphate buffered saline (PBS) supplemented with 0.1 M sodium bicarbonate at a 1.2:1 molar ratio of dye to protein. Excess dye was removed via 7 kDa desalting columns (Zeba Spin, ThermoFisher) and validated via thin-layer chromatography.

Pharmacokinetic and biodistribution in metastatic ovarian cancer model: B6C3F1 mice were inoculated with firefly luciferase-expressing OV2944-HM1 (HM-1) cells through intraperitoneal (i.p.) injection of 106 cells in PBS. Two weeks after tumor inoculation, mice were injected with NPs containing fluorescently labeled lipid (DOPE-630/650) and IL-12-ICG. The same IL-12-ICG was used for all groups to avoid labeling efficiency differences and groups were dosed intraperitoneally with 20 μg of IL-12. In vivo tumor radiant efficiency was measured on an In vivo Imaging System (IVIS, Perkin Elmer) by imaging the mice i.p. region and serum was collected via check bleeds. After the final timepoint (4 hours or 24 hours), mice were euthanized and organs were removed and had their NP and IL-12 radiant efficiency measured ex vivo on IVIS. Data were analyzed using Living Image software. Background fluorescence measurements were made for each organ based on signal from mice treated with PBS. Main tumor tissue was considered the omental adipose tissue which is the primary metastatic niche of ovarian cancer.³⁹ For correlation analysis, the weight-normalized bioluminescence flux (p/s/g) and radiant efficiency ([p/s]/[u W/cm²]/g) for each organ (excluding main tumor) were analyzed on Graphpad Prism 9 for their correlation via the Pearson's coefficient. Recovered fluorescence efficiency was calculated as described previously.⁴⁰ Serum cytokine levels were measured via ELISA (Peprotec).

Efficacy studies with metastatic ovarian cancer model: B6C3F1 mice were inoculated intraperitoneally with 106 cells of either HM-1-luc. One week after inoculation, treatment was initiated as indicated on each figure. All treatments received the same IL-12 dose. For combination with immune checkpoint inhibitors, mice received 250 μg of anti-PD1 antibody (clone 29F.1A12, BioXCell) and 100 μg of anti-CLTA4 antibody (clone CD152, BioXCell) i.p. one day after treatment with IL-12 constructs. Mice weight was tracked daily after treatments for signs of toxicity. For HM-1-luc tumors, bioluminescence was measured on a IVIS 10 minutes after i.p. injection of 3 μg of D-luciferin sodium salt (GoldBio) for 30 days after tumor inoculation or as needed to track tumor burden.

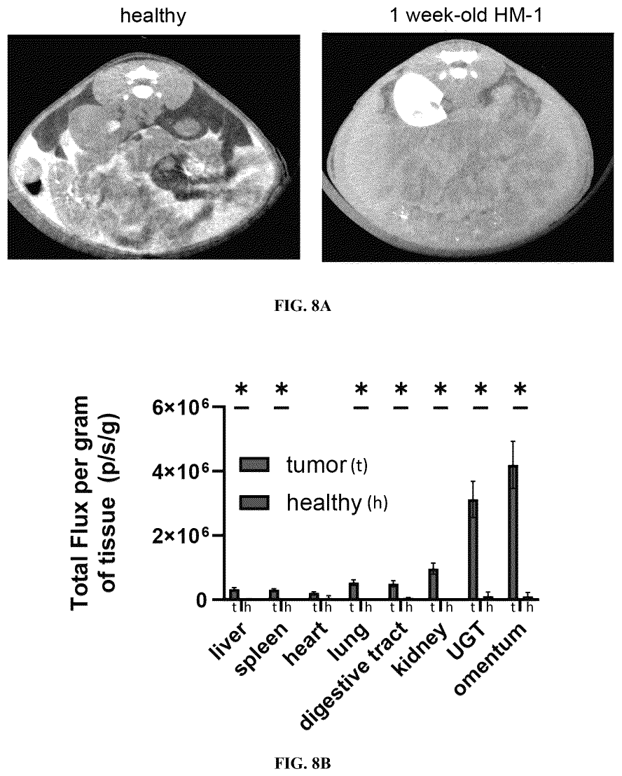

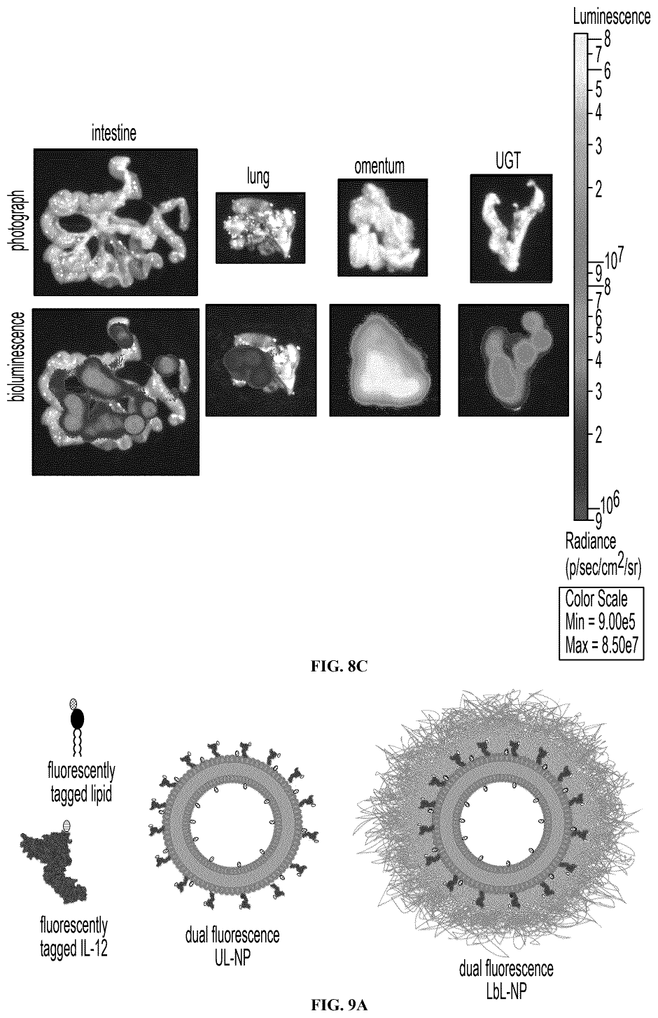

First, the model was briefly characterized and found that the tumor-bearing mice had significantly higher clearance from the intraperitoneal (i.p.) cavity based on micro computed tomography (micro-CT) scans which demonstrated rapid kidney accumulation of injected contrast agents (FIG. 8A). Such rapid clearance indicated the need of a delivery system to rapidly associated with tumor tissue in the i.p. cavity. Within 14 days following i.p. inoculation of luciferase-expressing HM-1 cells (luc-HM-1), bioluminescence imaging (BLI) revealed established tumor nodules dispersed across all tissues of the i.p. cavity, with a preference for the omentum and upper genital tract (UGT) tissues as is clinically observed with metastatic OC (FIG. 8B).²³ Analysis of organs showed clear formation of solid nodular tumor metastasis 7 days after inoculation (FIG. 8C). Overall, these results explain the previously observed difficulty in treating this model with standard-of-care cisplatin-based chemotherapeutics.²⁴

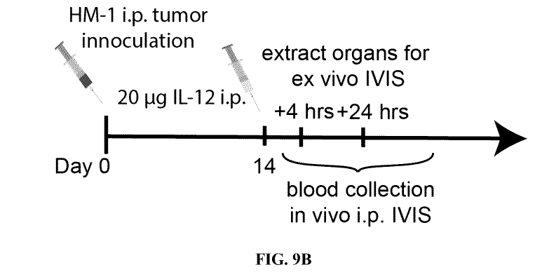

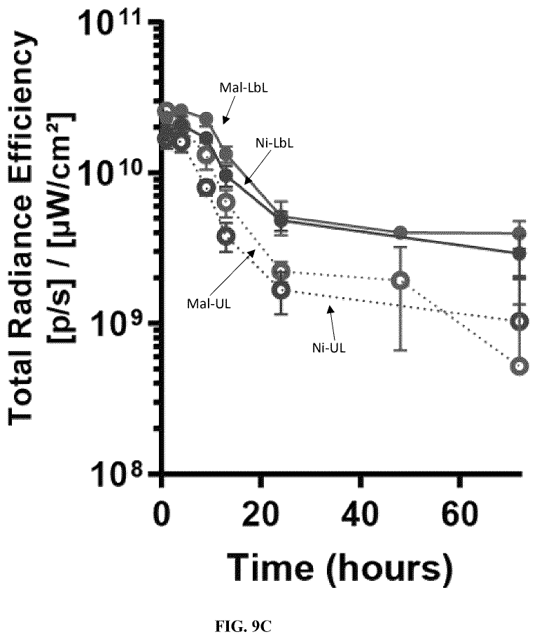

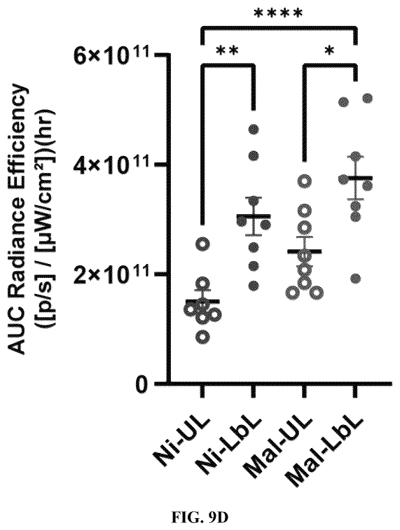

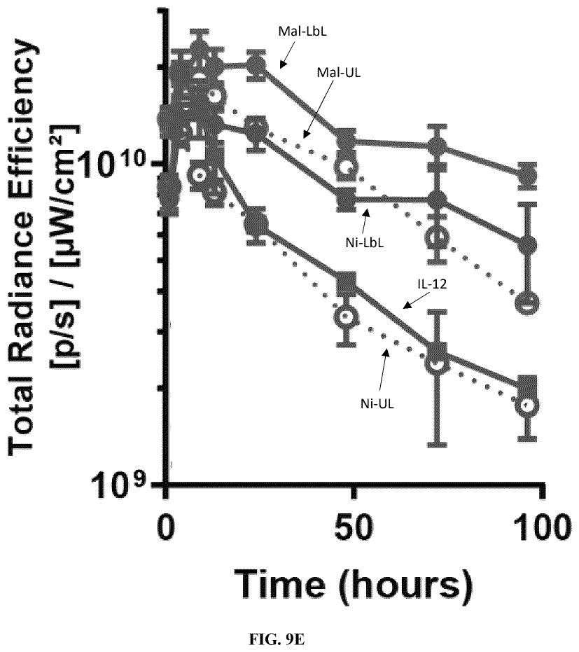

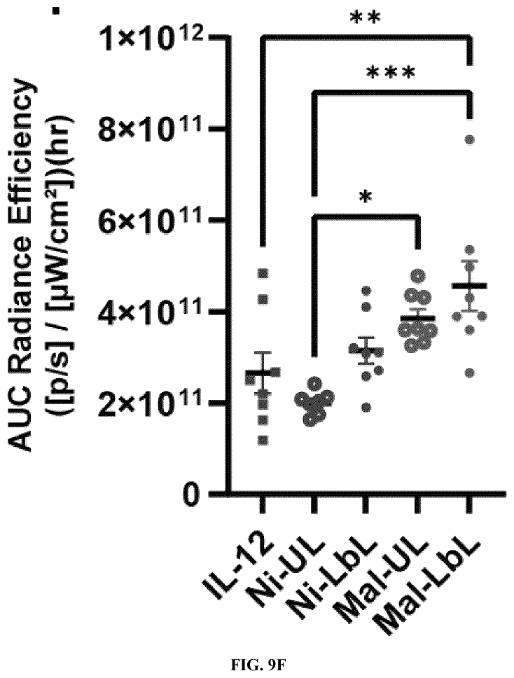

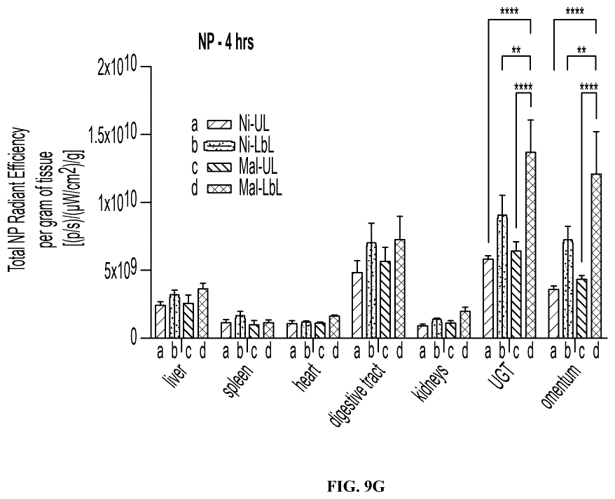

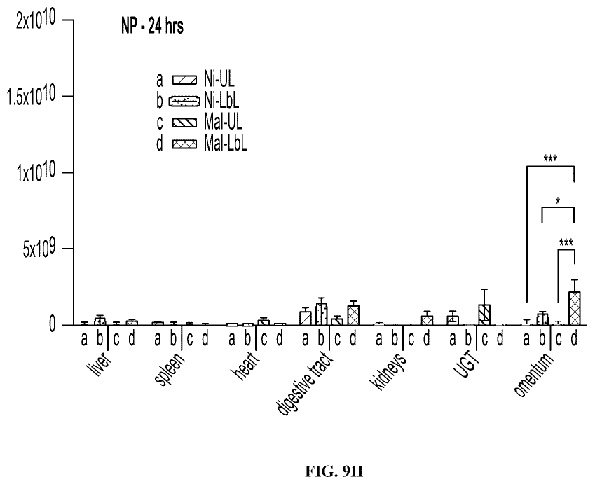

First, the pharmacokinetics (PK) of clearance of the LbL-coated or bare NPs and their IL-12 cargo in the i.p. space following administration were characterized. IL-12 was fluorescently tagged and used fluorescently labeled lipids in the NP formulations (FIG. 9A), and applied whole-animal fluorescence imaging to independently quantify levels of the NPs and IL-12 over time. Two weeks after tumor implantation, mice were dosed with the varied IL-12 constructs i.p. and blood was collected (FIG. 9B). These measurements revealed that LbL-coated NPs exhibited a longer lifetime in the i.p. space compared to uncoated particles (FIGS. 9C-9D). While the mode of IL-12 binding to the particles had no impact on NP PK, both the conjugation chemistry and LbL coating impact the rate of IL-12 clearance (FIGS. 9E-9F). Ni-histag-associated IL-12 delivered on unlayered particles cleared from the i.p. space at a rate indistinguishable from freely injected cytokine, suggesting rapid dissociation of IL-12 from these particles in vivo. LbL coating of Ni-histag particles prolonged the persistence of IL-12, but interestingly covalently conjugated IL-12 on unlayered NPs had a similar clearance rate despite the fact that Mal-UL particles are cleared more rapidly than Ni-LbL particles (FIGS. 9C-9F). Mal-LbL NPs led to the slowest clearance of IL-12 from the i.p. cavity, suggesting the importance of both the LbL coating to promote binding/retention on tumor cells and covalent conjugation of the cytokine to avoid premature release into the ascites fluid.

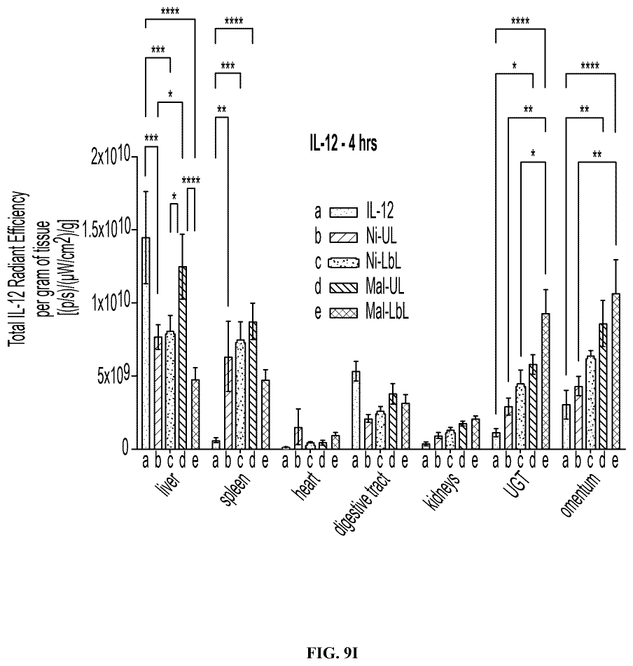

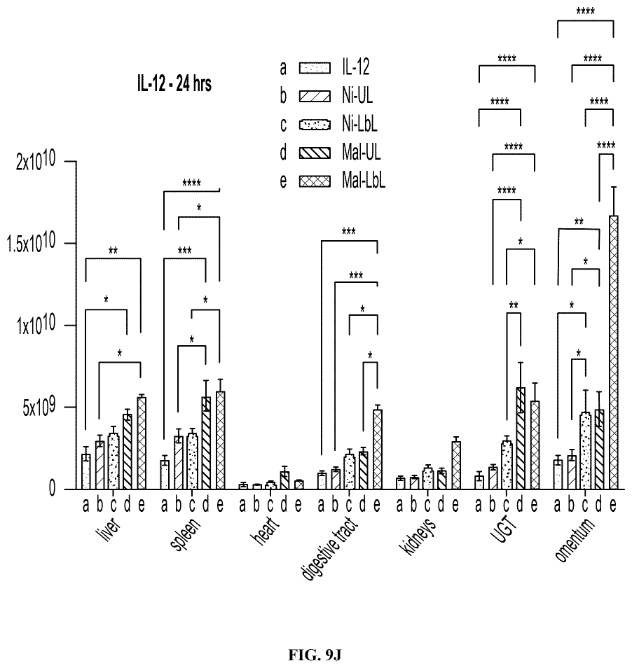

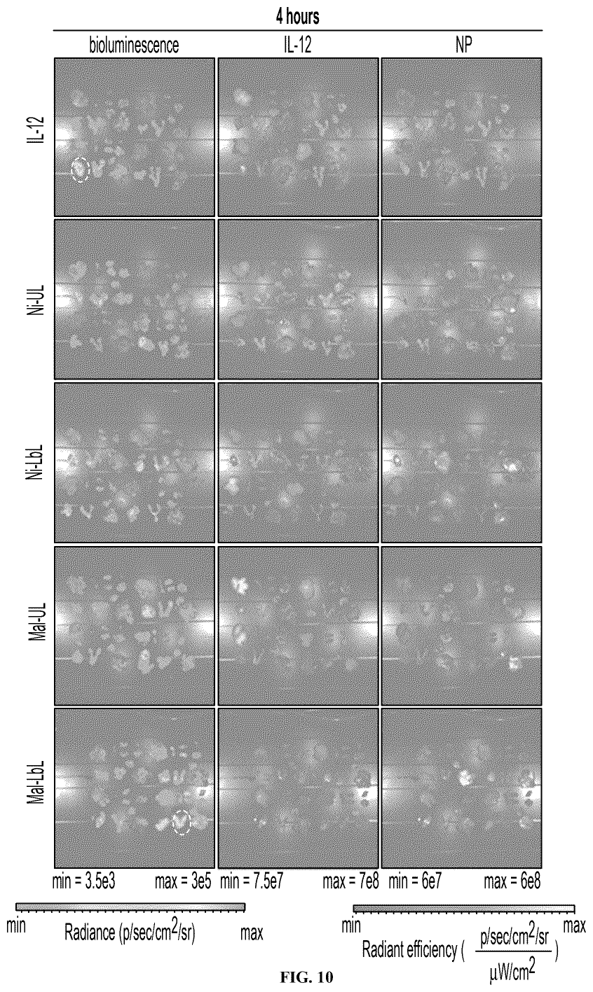

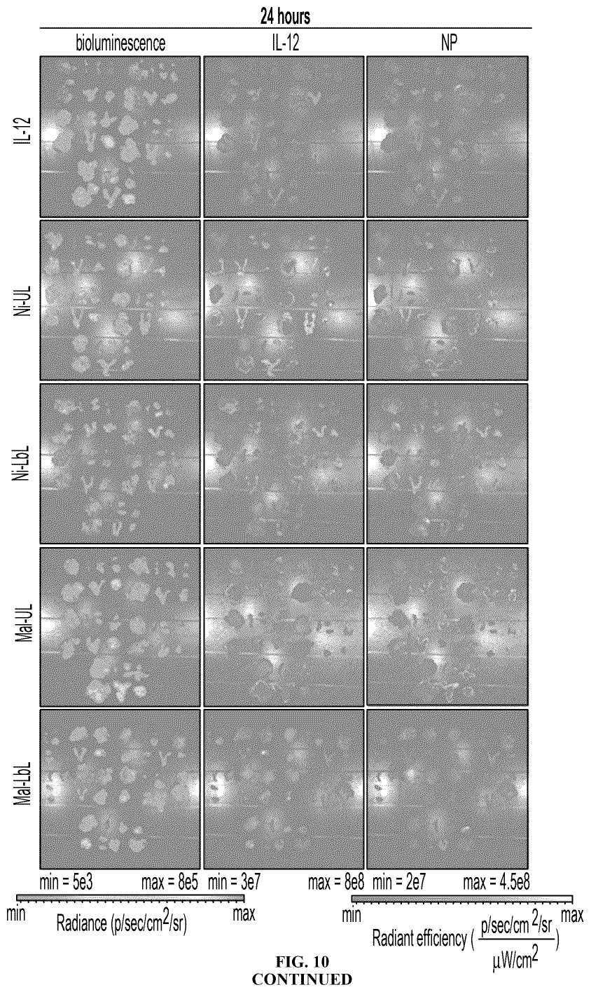

Organ biodistribution of NPs demonstrated that the LbL coating promoted NP association with the primary metastatic niche tissues (UGT and omentum) 4 hours after dosing. (FIG. 9G, FIG. 10). NP fluorescence decayed almost completely 24 hours after dosing with residual fluorescence present in the omentum for the Mal-LbL treated group (FIG. 9H). Analysis of IL-12 fluorescence 4 hours after dosing (FIG. 9I) showed that LbL coated NPs or Ni-UL had significantly lower liver IL-12 accumulation compared to the free cytokine or Mal-UL. Mal-LbL NPs were able to effectively deliver IL-12 to both UGT and omentum compared to free IL-12 indicating rapid accumulation of the cytokine in tumor tissue. The greater systemic retention of IL-12 in the Mal-LbL format was apparent 24 hours after dosing (FIG. 9J) where IL-12 fluorescence was elevated compared to other NP constructs in most tissues analyzed. This increased retention combined with the targeting properties of the LbL coating promoted a five-fold and ten-fold increase in the IL-12 levels in the UGT and omentum tissues, respectively.

Having established that Mal-LbL NPs could target metastatic OC, the tumor distribution of each construct was evaluated by analyzing sections of fresh frozen cryogenic omental tissue via confocal microscopy.

Cryogenic freezing of omentum tumors. Omentum tissue from the biodistribution study was added to optimal cutting temperature (OCT) compound and rapidly frozen in cryomolds using isopentane with dry ice. Samples were sectioned in 10 μm slices on a microtome-cryostat onto Tissue Path Superfrost Plus Gold Slides (Fisherbrand) and stored in −80° C. For staining, slides were rapidly fixed with ice-cold 4% methanol free formaldehyde for 10 minutes then washed with PBS and blocked with 10% goat serum for 1 hour. The samples were then incubated with PE anti-IL-12/IL-23 p40 antibody (Biolegend) overnight at 4° C. in 1% bovine serum albumin (BSA) PBS buffer. After overnight incubation, Hoechst 33342 (ThermoFisher) and WGA-alexafluor488 (ThermoFisher) were added and allowed to incubate for 30 minutes at room temperature. Samples were then washed with PBS then were closed with a coverslip with ProLong Gold (ThermoFisher) and stored at 4° C. after drying. Slides were imaged on a Olympus FV1200 Laser Scanning Confocal Microscope.

It was evident that while both Ni-Lbl and Mal-LbL presented with NP signal 24 hours after dosing, Mal-LbL enabled the greatest accumulation of IL-12 within the tumor (FIGS. 11A-11E). Analysis of Mal-LbL treated tumors under higher magnification indicated that Mal-LbL NPs were able to efficiently penetrate tumor tissue (FIG. 12A). Furthermore, it was observed that the Mal-LbL NPs accumulate on the outside of cancer cells as well as appear to diffusely stain the cell membrane, indicating lipid insertion into cell membranes (FIGS. 12B-12C). These results provide the first in vivo evidence of the altered cell surface localization enabled by the PLE coating in LbL-NPs.

### Example 5: Covalent Conjugation and Polyelectrolyte Coating Improve IL-12 Mediated Immunotherapy of Metastatic Ovarian Cancer

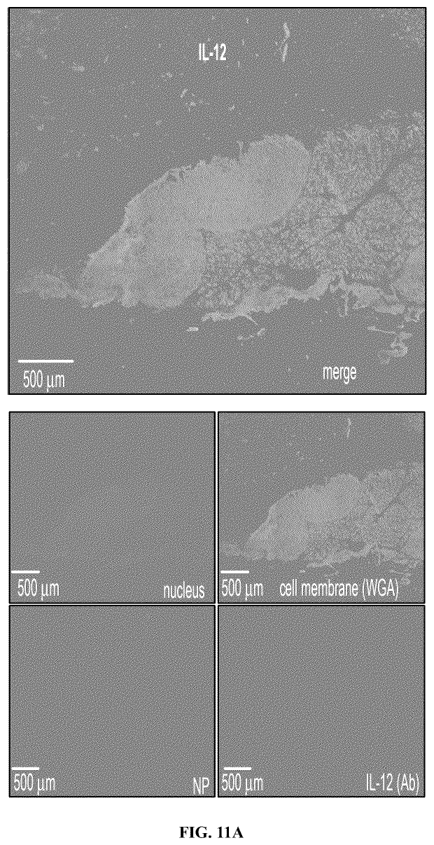

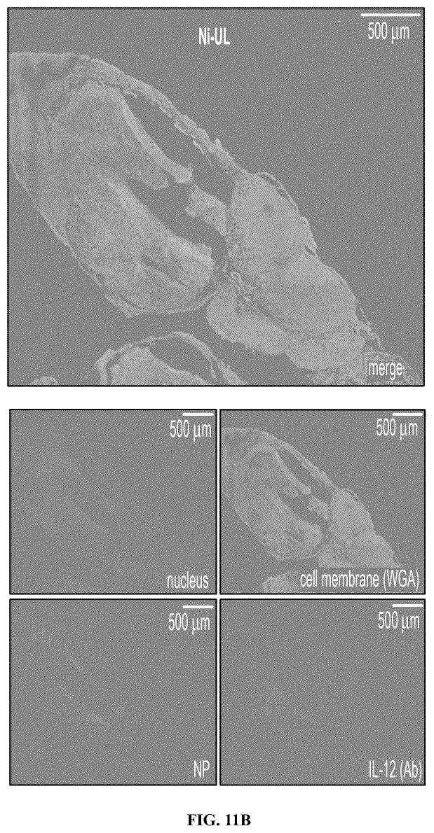

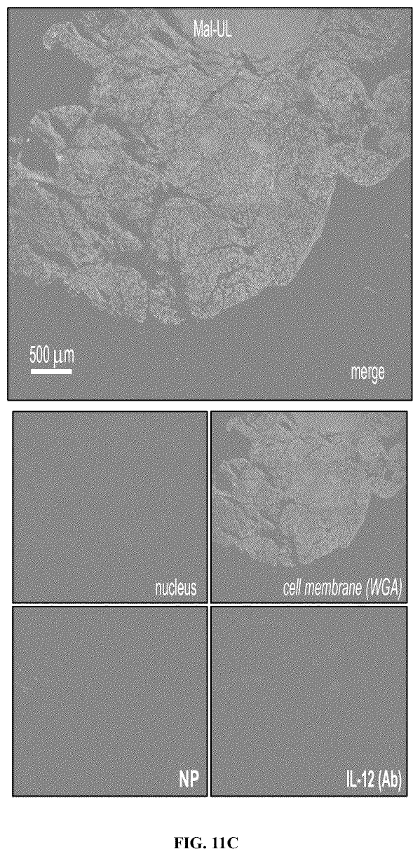

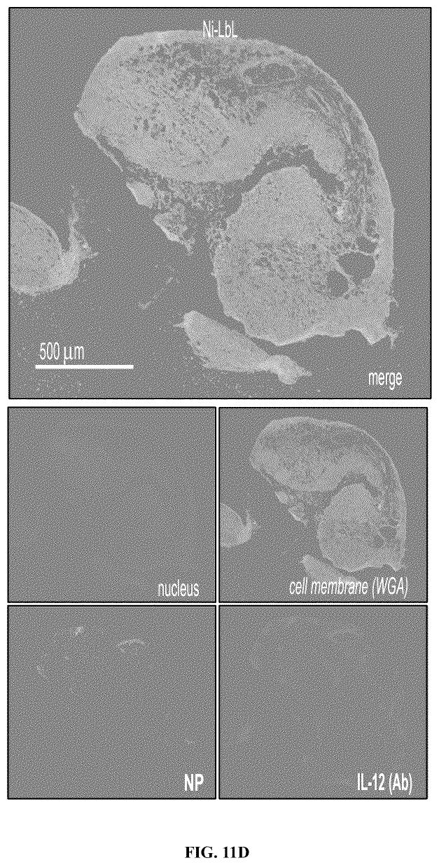

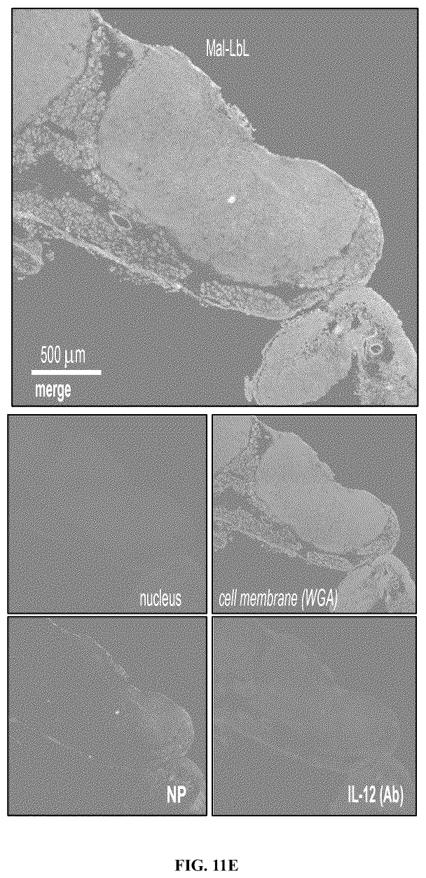

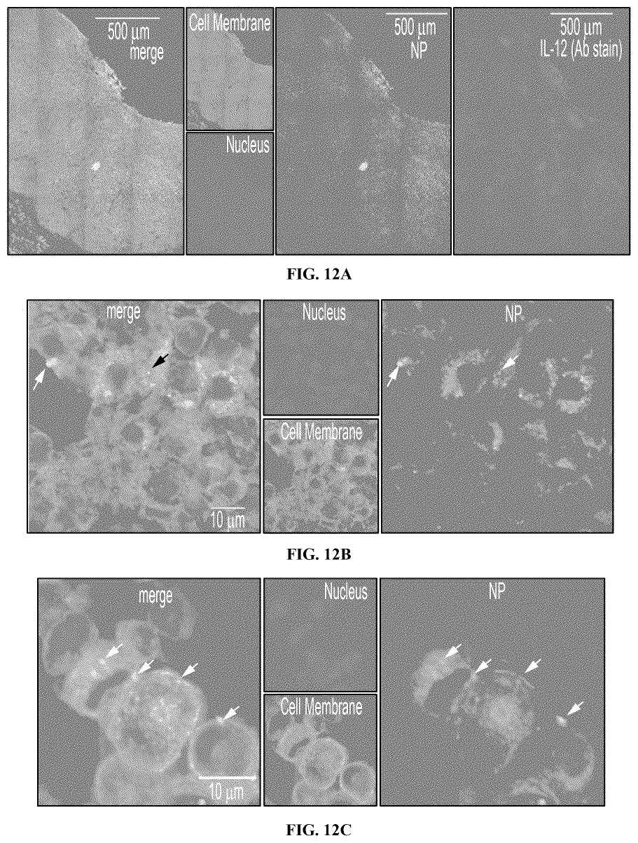

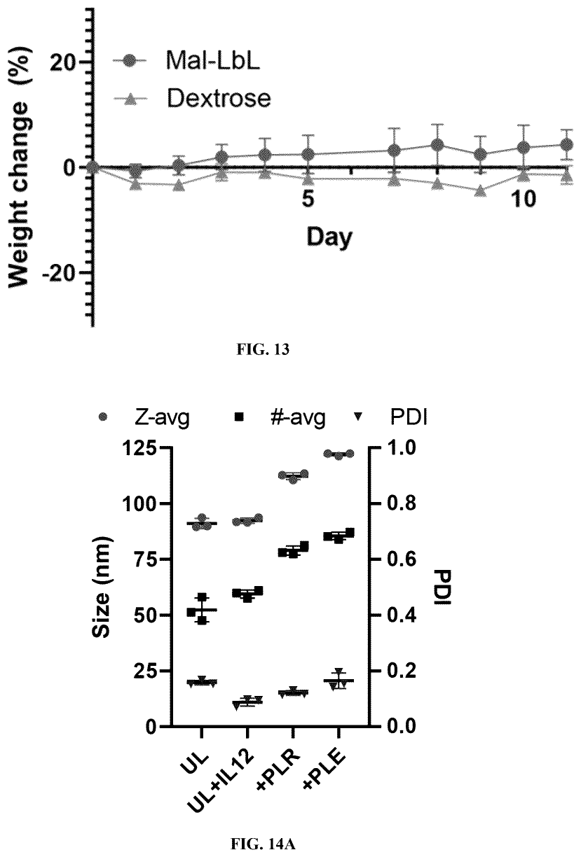

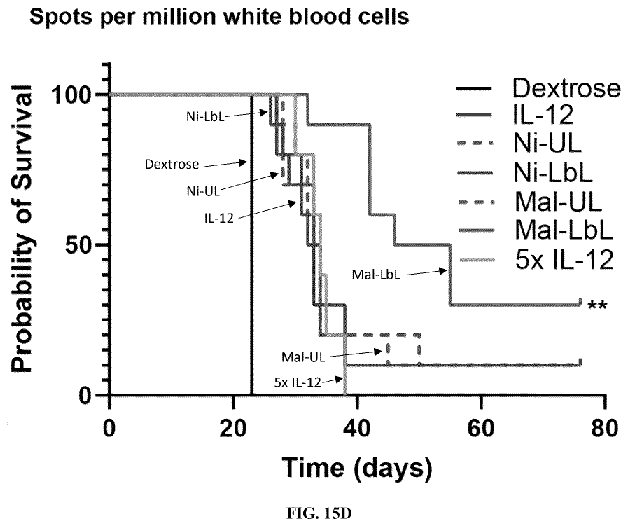

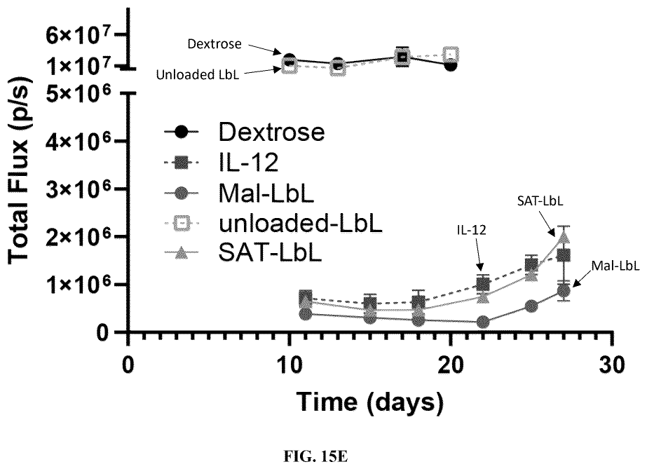

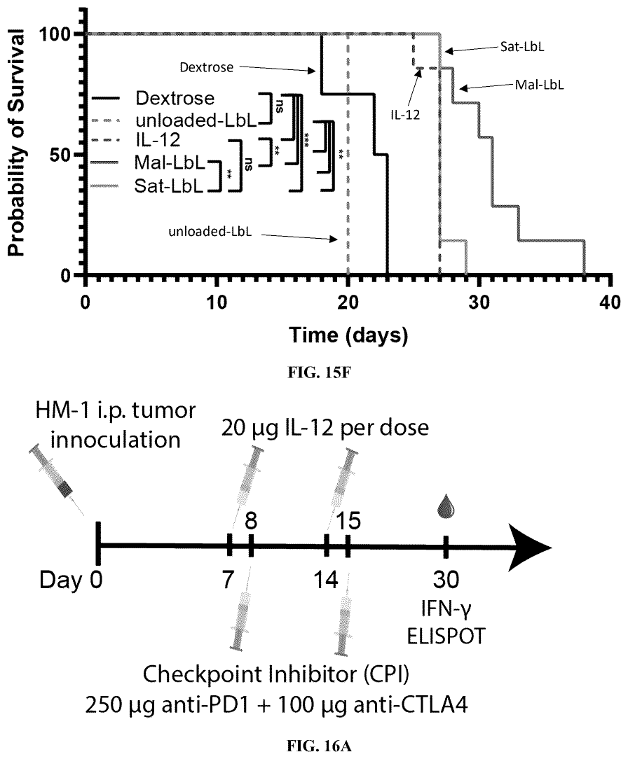

Next, the impact of increased tumor targeting achieved by Mal-LbL NPs on the therapeutic efficacy of IL-12 therapy was assessed. HM-1-luc tumor-bearing mice were treated with 20 μg of IL-12 as freely administered cytokine or nanoparticle formulations at days 7 and 14, or a 5× higher dose of the free cytokine (to determine whether higher dosing of free cytokine could compensate for its rapid clearance, FIG. 15A). Three days following the first dose, a dramatic drop in tumor luciferase signal was observed for all treated animals, but tumor signals began to rebound for all of the treatment groups except Mal-LbL NPs by day 24 (FIG. 15B). All of the animals receiving free IL-12 (including the 5× dose), histag-anchored IL-12 NPs, or unlayered covalent-anchored IL-12 NPs showed an increase in median survival to ˜33 days, compared to 23 days for the untreated tumors, but tumors relapsed leading to death of the vast majority of the animals in these groups (FIG. 15C). By contrast, Mal-LbL NP treatment led to a further increase in median survival to 44 days, with ˜30% of the animals eliciting complete responses (FIG. 15D). IFN-γ ELISPOT analysis of peripheral blood mononuclear cells (PBMCs) on day 30 via revealed a stronger tumor-specific T cell response induced by the Mal-LbL NP treatment compared to all other groups (FIG. 15B). When mice that rejected their tumors were rechallenged i.p. with one-third the tumor cell dose, all mice had rapid tumor cell clearance and survived, indicating successful development of a memory response. This treatment schedule with NPs bearing covalently-anchored IL-12 showed no signs of toxicity as animal showed no sign of weight loss following dosing (FIG. 13).

LbL-NPs can accumulate on cell surfaces which could induce changes in cellular phenotype or mediate an immune response. To better understand the mechanism of improved tumor treatment, LbL-NPs devoid of IL-12 (unloaded-LbL) were generated. Further, as it had been observed in vitro that Mal-LbL NPs could undergo lipid exchange to release IL-12, a maleimide-conjugated LbL-NP composition was generated to minimize lipid exchange and to test if slow release of IL-12 from the particles in vivo was important for therapeutic efficacy.

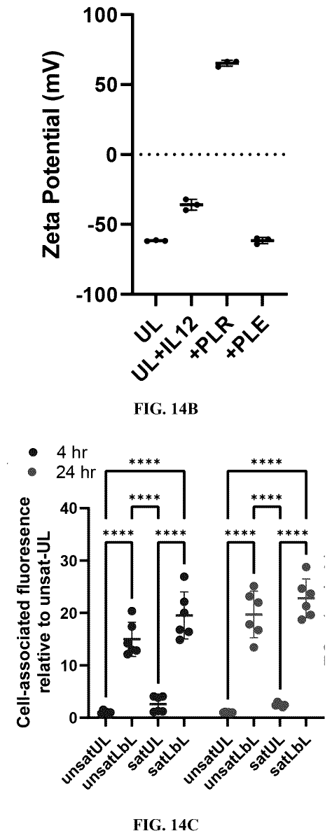

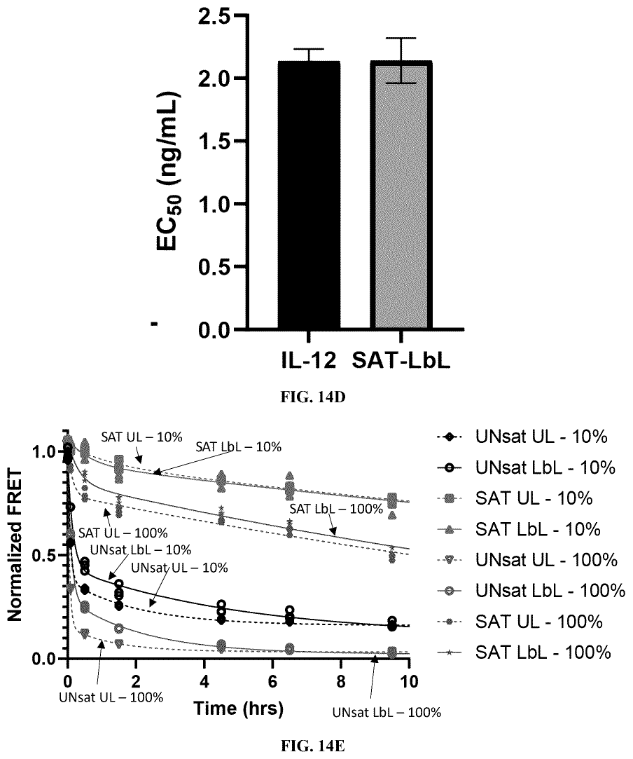

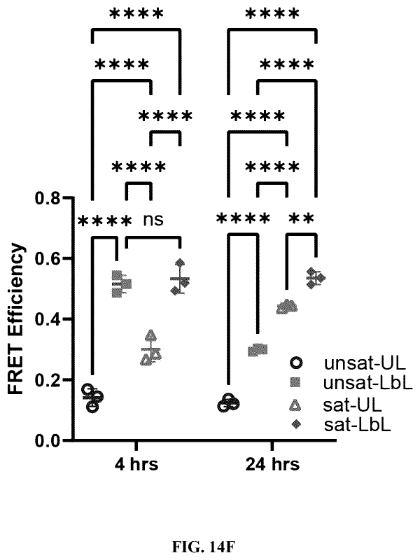

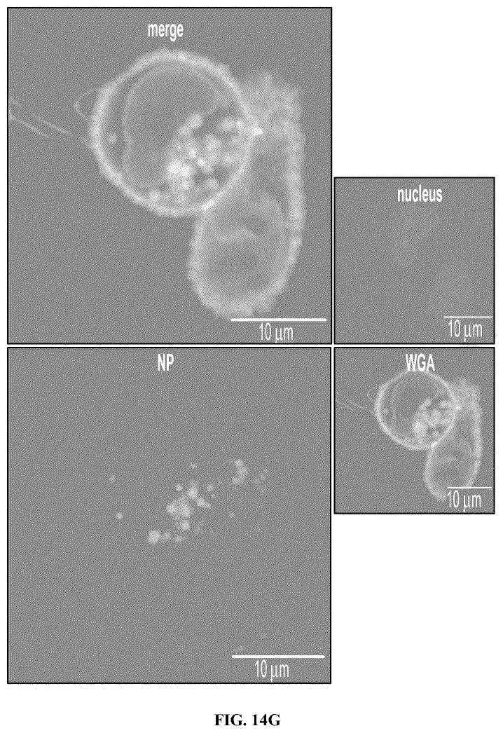

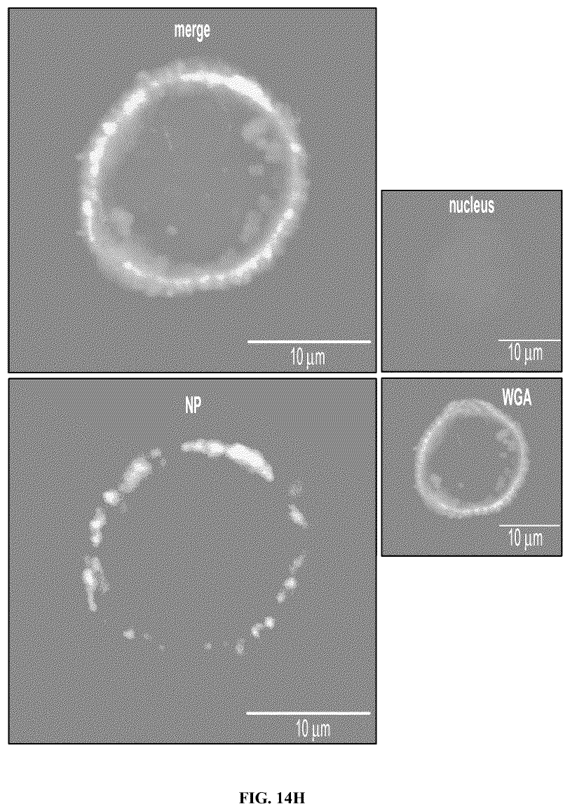

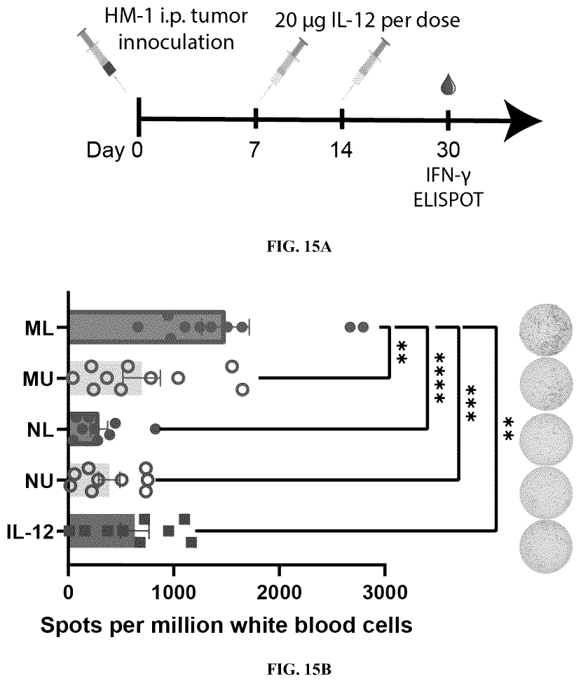

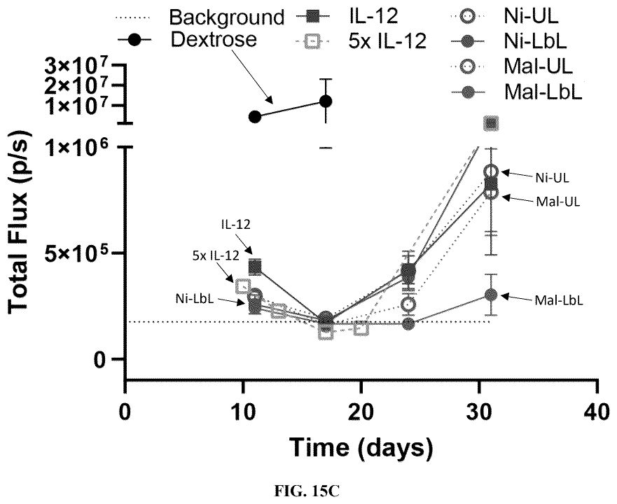

To this end, liposomes incorporating only fully saturated lipids were prepared. These fully saturated LbL-NPs (SAT-LbL) were prepared with the same size and zeta potential as Mal-LbL particles, and showed identical binding to HM-1 cells and IL-12 reporter cell activation in vitro (FIGS. 14A-14D). Furthermore, when high density FRET NPs were generated, SAT NPs were confirmed to be more resistant to lipid release in serum (FIG. 14E) as well as when associated with HM-1 cells (FIG. 14F). Similar to unsaturated formulations, unlayered saturated NP were endocytosed (FIG. 14G) whereas SAT-LbL accumulated on the cell surface (FIG. 14H) 4 hours after dosing to HM-1 cells in vitro.

To compare the therapeutic efficacy of Mal-LbL to that of unloaded-LbL (dosed with a 20 ug IL-12 equivalent mass of lipids) and SAT-LbL, a single 20 μg of IL-12 one week after i.p. inoculation of HM-1-luc tumor cells was administered. Unloaded-LbL NPs did not induce any therapeutic benefit compared to control dextrose-treated mice, while SAT-LbL NPs elicited the same kinetic of tumor relapse and survival benefit as free IL-12 (FIGS. 15E-15F). By contrast, Mal-LbL NPs showed no tumor recurrence at day 22 and extended median survival by an additional 15% beyond the other two IL-12 treatments, indicating that the lipid-exchange properties of Mal-LbL participated in the improved therapeutic benefit seen with this formulation.

### Example 6: Polyelectrolyte Coated IL-12 NPs Synergize with Immune Checkpoint Inhibitors Leading to Curative Treatment of Metastatic Ovarian Cancer

One of IL-12's main effects is to induce expression of IFN-γ in T cells and NK cells, which in turn will upregulate expression of PD-L1 on tumor cells, a phenomenon termed adaptive resistance.³⁰ Thus, it was hypothesized that LbL-NP treatment should synergize with anti-PD-1 checkpoint inhibitors (CPIs) that are approved for treatment of diverse cancers, but which have failed to demonstrate efficacy as monotherapies in ovarian cancer.³¹ Further, the combination of anti-PD-1 with anti-CTLA-4, a second CPI, can further augment T cell-mediated anti-tumor immunity and is approved for treatment of several cancers, though this combination elicited very minor increases in overall survival of ovarian cancer patients compared to anti-PD-1 alone.³²

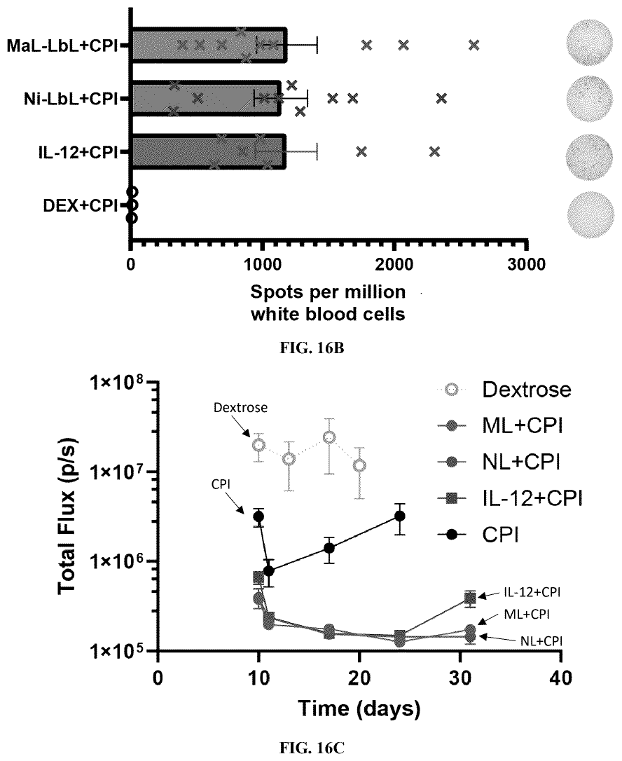

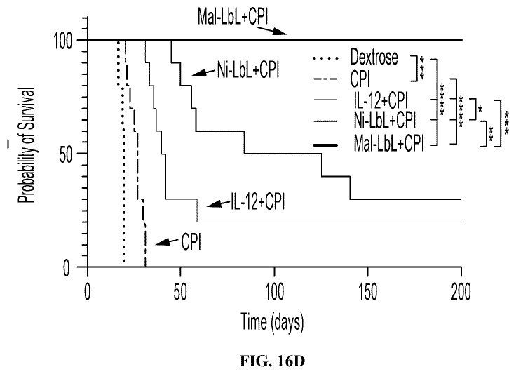

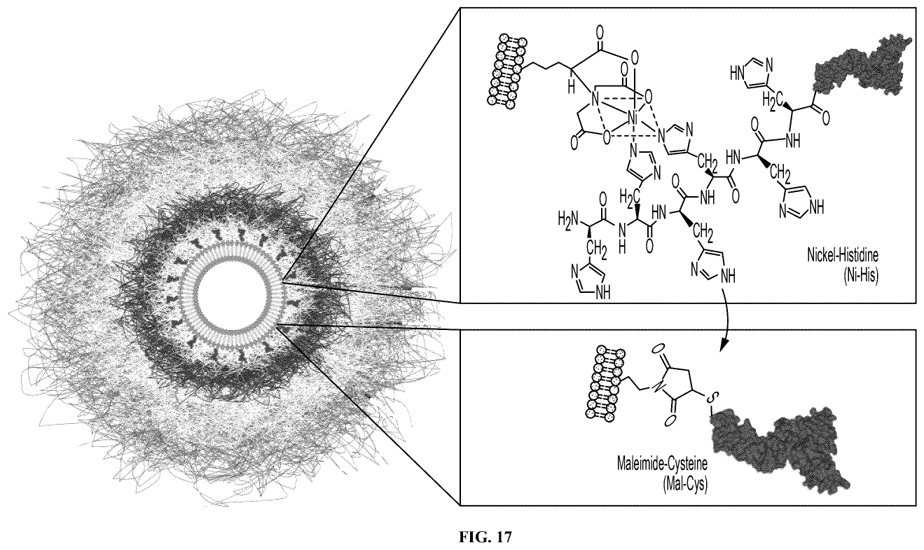

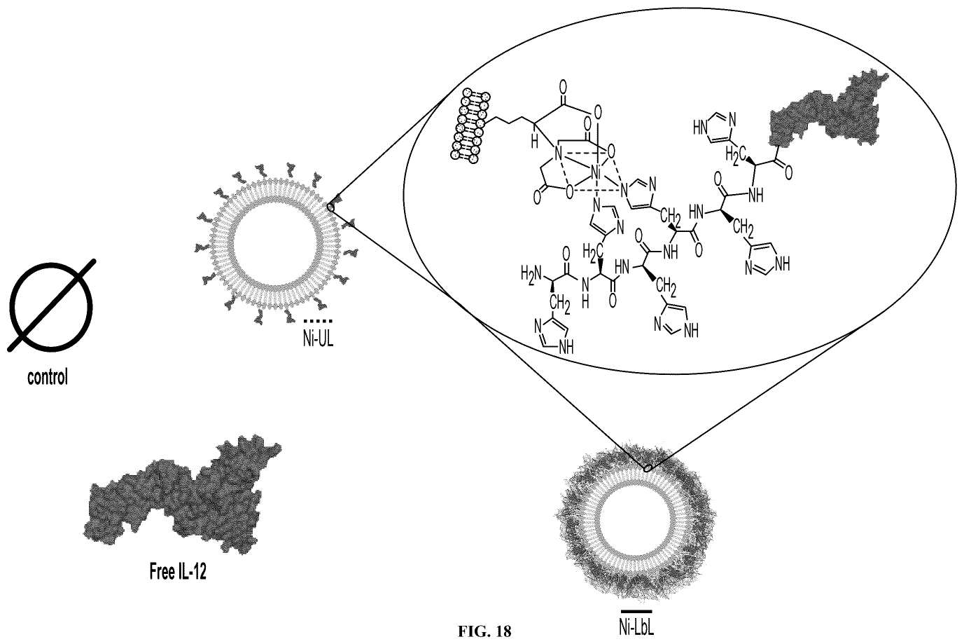

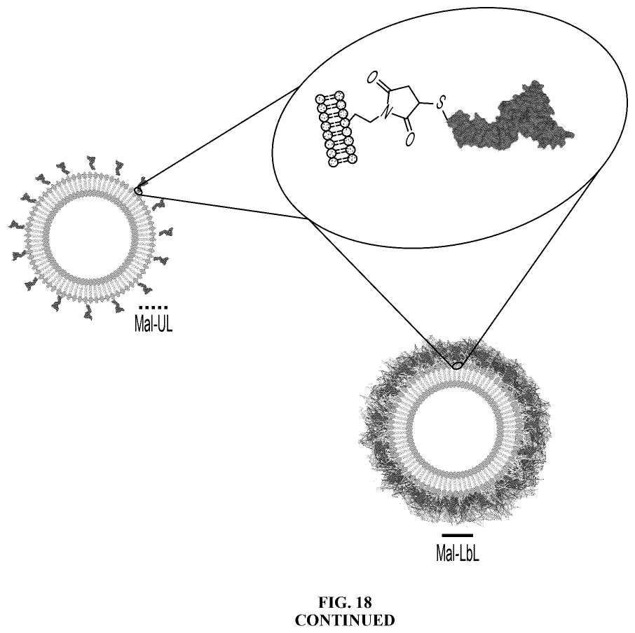

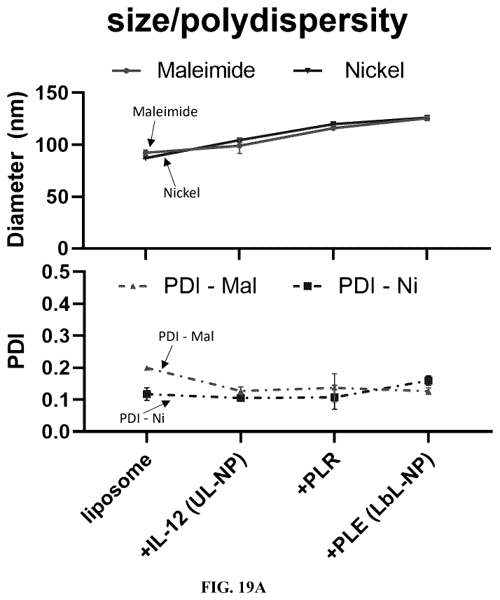

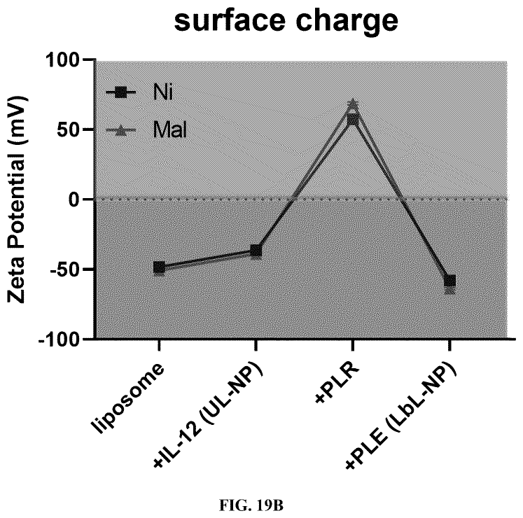

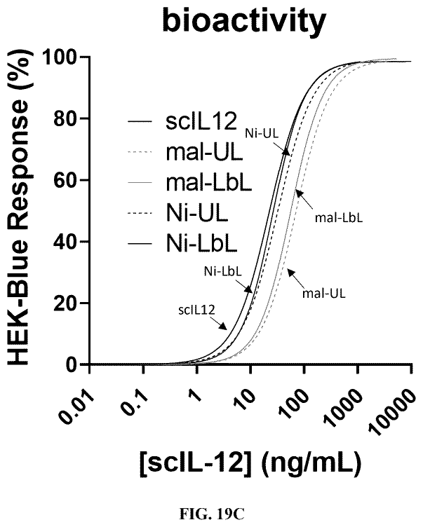

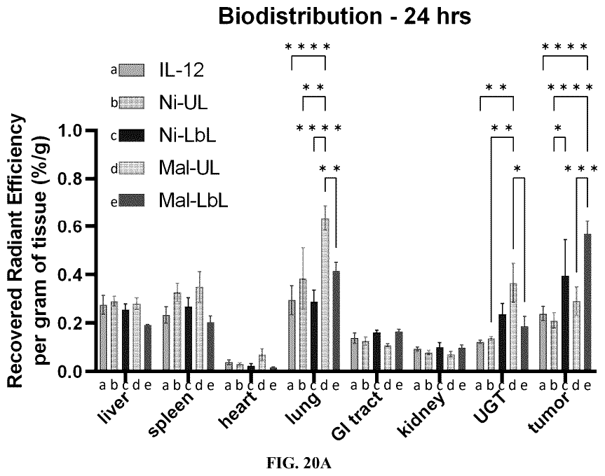

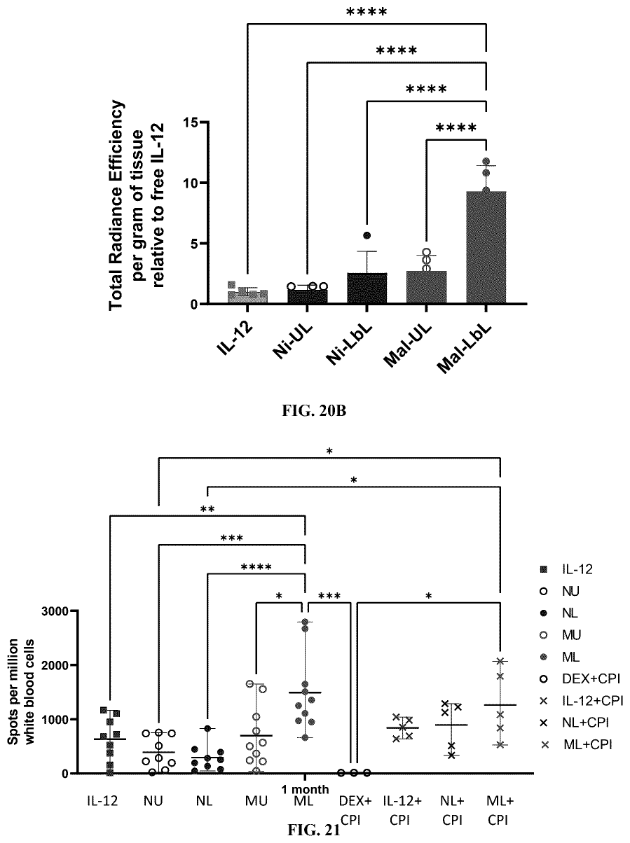

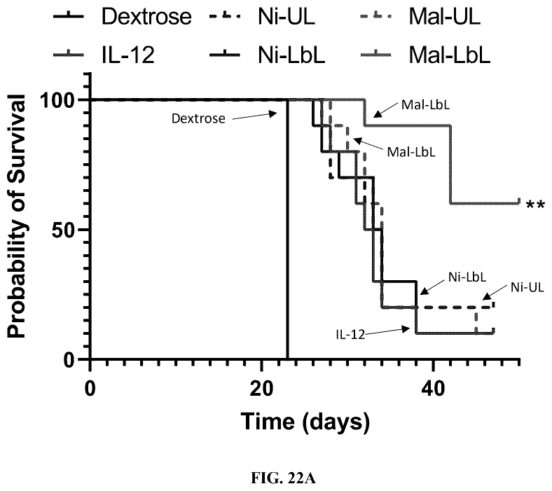

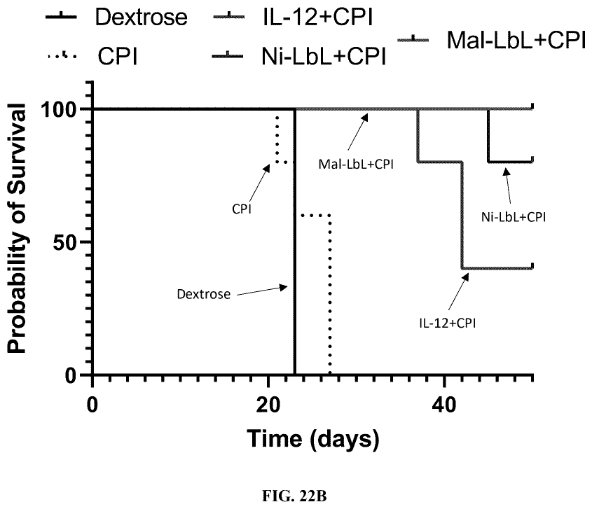

To determine whether LbL-NP delivery of IL-12 could augment the efficacy of CPI treatment, treatment of HM-1-luc tumors with systemic anti-PD-1+anti-CTLA-4 alone or combined with two weekly doses of IL-12 in free or NP form (FIG. 16A) was evaluated.

Mice received 250 μg of anti-PD1 antibody (clone 29F.1A12, BioXCell) and 100 μg of anti-CLTA4 antibody (clone CD152, BioXCell) i.p. one day after treatment with IL-12 constructs. Mice weight was tracked daily after treatments for signs of toxicity. For HM-1-luc tumors, bioluminescence was measured on a IVIS 10 minutes after i.p. injection of 3 μg of D-luciferin sodium salt (GoldBio) for 30 days after tumor inoculation or as needed to track tumor burden.

Combination of IL-12—in free of NP form—with CPIs enabled robust anti-tumor immune response as measured via IFN-γ ELISPOT for all groups, albeit CPI only showed no appreciable levels of tumor-reactive white blood cells (FIG. 16B). Analysis of tumor burden via BLI clearly showed that CPIs alone could only mildly and transiently control tumor growth (FIG. 16C). On the other hand, there was a marked synergism when IL-12 was included in the treatment with CPI, with both Mal-LbL and Ni-LbL particles showing no measurable levels of tumor BLI one month after tumor inoculation. Indeed, CPIs alone had only marginal therapeutic benefit, similar to what has been observed with OC in the clinic whereas free IL-12 with CPI showed some efficacy, curing 20% of mice (FIG. 16D).³³ Strikingly, however, Mal-LbL showed a remarkable synergistic effect, achieving 100% cures with this treatment schedule. Ni-LbL also synergized with CPI treatment, showing a significant extension in survival compared to free IL-12 and CPI but ultimately resulted in 30% of mice cured. Mal-LbL-treated mice survived rechallenge with one third (5/5) and the full inoculation dose of HM-1 (5/5) showing rapid tumor cell rejection after i.p. injection, demonstrating induction of a strong memory response.

### Example 8: Alternative Particle Formulations

Particles comprising one or more of DOPE-Mal, DOPE-NHS, DOPE-azide, DOPE-DBCO, and DOPE-TCO as the first lipid, one or more of HSPC, DPPC, DSPE, DOPE, DPPE, POPE, egg phosphatidylcholine, and soy phosphatidylcholine as second lipids, one or more of campesterol, desmosterol, stigmasterol, lanosterol, and sitosterol as a sterol, and one or more of DSPG, DPPG, DOPG, POPI, DSPI, DOPI, egg phosphatidylglycerol, and soy phosphatidylglycerol as third lipids are prepared according to the procedure of Example 1. These particles are expected to demonstrate the beneficial properties of the particles of Examples 1-7.

### Example 8: Antibody Particle Formulations

Particles comprising in place of IL-12 one or more of anti-PD1 antibodies, anti-PDL1 antibodies, anti-CTLA4 antibodies, anti-TIM-3 antibodies, anti-LAG-3 antibodies, anti-NKG2A antibodies, anti-CD73 antibodies, anti-A2aR antibodies, anti-B7-H3 antibodies, anti-B7-H4 antibodies are prepared according to the procedure of Example 1. These particles are expected to demonstrate the beneficial properties of the particles of Examples 1-7.

### Example 9: Additional Cytokine Particle Formulations

Particles comprising in place of IL-2 one or more of IL-2, IFN-γ, IFN-α, IFN-β, IL-15, IL-15SA, IL-18, TNF-α, IL-10, IL-8, TRAIL, and FLT3L3 are prepared according to the procedure of Example 1. These particles are expected to demonstrate the beneficial properties of the particles of Examples 1-7.

### REFERENCES

1. Esfahani, K. et al. A Review of Cancer Immunotherapy: From the Past, to the Present, to the Future. Current Oncology 27, 87-97 (2020).
2. Arora, T., Mullangi, S. & Lekkala, M. R. Ovarian Cancer. (2023).
3. Chardin, L. & Leary, A. Immunotherapy in Ovarian Cancer: Thinking Beyond PD-1/PD-L1. Front Oncol 11, (2021).
4. Anderson, K. G., Stromnes, I. M. & Greenberg, P. D. Obstacles Posed by the Tumor Microenvironment to T cell Activity: A Case for Synergistic Therapies. Cancer Cell 31, 311-325 (2017).
5. Lheureux, S., Braunstein, M. & Oza, A. M. Epithelial ovarian cancer: Evolution of management in the era of precision medicine. CA Cancer J Clin caac.21559 (2019) doi: 10.3322/caac.21559.
6. Matulonis, U. A. et al. Efficacy and Safety of Mirvetuximab Soravtansine in Patients With Platinum-Resistant Ovarian Cancer With High Folate Receptor Alpha Expression: Results From the SORAYA Study. Journal of Clinical Oncology 41, 2436-2445 (2023).
7. Pires, I. S., Hammond, P. T. & Irvine, D. J. Engineering Strategies for Immunomodulatory Cytokine Therapies: Challenges and Clinical Progress. Adv Ther (Weinh) 4, 2100035 (2021).
8. Agarwal, Y. et al. Intratumourally injected alum-tethered cytokines elicit potent and safer local and systemic anticancer immunity. Nat Biomed Eng 6, 129-143 (2022).
9. Correa, S. et al. Tuning Nanoparticle Interactions with Ovarian Cancer through Layer-by-Layer Modification of Surface Chemistry. ACS Nano 14, 2224-2237 (2020).
10. Barberio, A. E. et al. Cancer Cell Coating Nanoparticles for Optimal Tumor-Specific Cytokine Delivery. ACS Nano 14, 11238-11253 (2020).
11. Barberio, A. E. et al. <scp>Layer-by-layer</scp>interleukin-12 nanoparticles drive a safe and effective response in ovarian tumors. Bioeng Transl Med 8, (2023).
12. Rüger, R., Müller, D., Fahr, A. & Kontermann, R. E. In vitro characterization of binding and stability of single-chain Fv Ni-NTA-liposomes. J Drug Target 14, 576-582 (2006).
13. Platt, V. et al. Influence of Multivalent Nitrilotriacetic Acid Lipid-Ligand Affinity on the Circulation Half-Life in Mice of a Liposome-Attached His 6-Protein. Bioconjug Chem 21, 892-902 (2010).
14. Mensah, L. B. et al. Layer-by-layer nanoparticles for novel delivery of cisplatin and PARP inhibitors for platinum-based drug resistance therapy in ovarian cancer. Bioeng Transl Med 4, (2019).
15. Tošić, I. et al. Lipidome-based Targeting of STAT3-driven Breast Cancer Cells Using Poly-<scp>1</scp>-glutamic Acid-coated Layer-by-Layer Nanoparticles. Mol Cancer Ther 20, 726-738 (2021).
16. Kong, S. et al. Synergistic combination therapy delivered via layer-by-layer nanoparticles induces solid tumor regression of ovarian cancer. Bioeng Transl Med 8, (2023).
17. Su, D. & Zhang, D. Linker Design Impacts Antibody-Drug Conjugate Pharmacokinetics and Efficacy via Modulating the Stability and Payload Release Efficiency. Front Pharmacol 12, (2021).
18. Ravasco, J. M. J. M., Faustino, H., Trindade, A. & Gois, P. M. P. Bioconjugation with Maleimides: A Useful Tool for Chemical Biology. Chemistry-A European Journal 25, 43-59 (2019).
19. Hashimoto, M., Niwa, O., Nitta, Y., Takeichi, M. & Yokoro, K. Unstable Expression of E-Cadherin Adhesion Molecules in Metastatic Ovarian Tumor Cells. Japanese Journal of Cancer Research 80, 459-463 (1989).
20. Liu, H. et al. Structure-based programming of lymph-node targeting in molecular vaccines. Nature 507, 519-522 (2014).
21. Kong, S. M., Costa, D. F., Jagielska, A., Van Vliet, K. J. & Hammond, P. T. Stiffness of targeted layer-by-layer nanoparticles impacts elimination half-life, tumor accumulation, and tumor penetration. Proceedings of the National Academy of Sciences 118, (2021).
22. Boehnke, N. et al. Massively parallel pooled screening reveals genomic determinants of nanoparticle delivery. Science (1979) 377, (2022).
23. Iwagoi, Y. et al. Omental metastasis as a predictive risk factor for unfavorable prognosis in patients with stage III-IV epithelial ovarian cancer. Int J Clin Oncol 26, 995-1004 (2021).
24. Toyoshima, M. et al. Generation of a syngeneic mouse model to study the intraperitoneal dissemination of ovarian cancer with in vivo luciferase imaging. Luminescence 24, 324-331 (2009).
25. Lee, G. et al. Lymphatic Uptake of Liposomes after Intraperitoneal Administration Primarily Occurs via the Diaphragmatic Lymphatics and is Dependent on Liposome Surface Properties. Mol Pharm 16, 4987-4999 (2019).
26. Sarfarazi, A. et al. Therapeutic delivery to the peritoneal lymphatics: Current understanding, potential treatment benefits and future prospects. Int J Pharm 567, 118456 (2019).
27. Slater, S. J., Ho, C., Taddeo, F. J., Kelly, M. B. & Stubbs, C. D. Contribution of hydrogen bonding to lipid-lipid interactions in membranes and the role of lipid order: Effects of cholesterol, increased phospholipid unsaturation, and ethanol. Biochemistry 32, 3714-3721 (1993).
28. Zhu, X. et al. Surface De-PEGylation Controls Nanoparticle-Mediated siRNA Delivery In Vitro and In vivo. Theranostics 7, 1990-2002 (2017).
29. Münter, R. et al. Dissociation of fluorescently labeled lipids from liposomes in biological environments challenges the interpretation of uptake studies. Nanoscale 10, 22720-22724 (2018).
30. Spranger, S. et al. Up-Regulation of PD-L1, IDO, and T regs in the Melanoma Tumor Microenvironment Is Driven by CD8+T Cells. Sci Transl Med 5, (2013).
31. Hamanishi, J. et al. Nivolumab Versus Gemcitabine or Pegylated Liposomal Doxorubicin for Patients With Platinum-Resistant Ovarian Cancer: Open-Label, Randomized Trial in Japan (NINJA). Journal of Clinical Oncology 39, 3671-3681 (2021).
32. Zamarin, D. et al. Randomized Phase II Trial of Nivolumab Versus Nivolumab and Ipilimumab for Recurrent or Persistent Ovarian Cancer: An NRG Oncology Study. Journal of Clinical Oncology 38, 1814-1823 (2020).
33. Zamarin, D. et al. Randomized Phase II Trial of Nivolumab Versus Nivolumab and Ipilimumab for Recurrent or Persistent Ovarian Cancer: An NRG Oncology Study. Journal of Clinical Oncology 38, 1814-1823 (2020).
34. Riley, R. S., June, C. H., Langer, R. & Mitchell, M. J. Delivery technologies for cancer immunotherapy. Nat Rev Drug Discov 18, 175-196 (2019).
35. Nguyen, K. G. et al. Localized Interleukin-12 for Cancer Immunotherapy. Front Immunol 11, (2020).
36. Champiat, S. et al. Intratumoral Immunotherapy: From Trial Design to Clinical Practice. Clinical Cancer Research 27, 665-679 (2021).
37. Chen, B.-M., Cheng, T.-L. & Roffler, S. R. Polyethylene Glycol Immunogenicity: Theoretical, Clinical, and Practical Aspects of Anti-Polyethylene Glycol Antibodies. ACS Nano 15, 14022-14048 (2021).
38. Edidin, M. Fluorescence Resonance Energy Transfer: Techniques for Measuring Molecular Conformation and Molecular Proximity. Curr Protoc Immunol 57, (2003).
39. Krishnan, V. et al. Omental macrophages secrete chemokine ligands that promote ovarian cancer colonization of the omentum via CCR1. Commun Biol 3, 524 (2020).
40. Correa, S. et al. Tuning Nanoparticle Interactions with Ovarian Cancer through Layer-by-Layer Modification of Surface Chemistry. ACS Nano 14, 2224-2237 (2020).

### INCORPORATION BY REFERENCE

The present application refers to various issued patent, published patent applications, scientific journal articles, and other publications, all of which are incorporated herein by reference. The details of one or more embodiments of the present disclosure are set forth herein. Other features, objects, and advantages of the present disclosure will be apparent from the Detailed Description, the Figures, the Examples, and the Claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A particle comprising:
(a) a liposome, wherein the liposome comprises at least one first lipid covalently bonded to at least one immunostimulatory agent; and the first lipid forms an outer surface of the liposome;
(b) a polymer coating, wherein the polymer coating comprises:
at least one layer including a polycation, wherein the polycation is non-covalently associated with the outer surface of the liposome; and
at least one layer including a polyanion, wherein the polyanion is non-covalently associated with the at least one polycation layer.

2. The particle of claim 1, wherein the liposome further comprises a second lipid; and the second lipid is a phospholipid selected from the group consisting of 1,2-distearoyl-sn-glycero-3-phosphocholine (DSPC), hydrogenated soy phosphatidylcholine (HSPC), 1,2-dipalmitoyl-sn-glycero-3-phosphocholine (DPPC), 1,2-distearoyl-sn-glycero-3-phosphoethanolamine (DSPE), 1,2-dioleoyl-sn-glycero-3-phosphoethanolamine (DOPE), 1,2-Dipalmitoyl-sn-glycero-3-phosphoethanolamine (DPPE), soy phosphatidylcholine, and egg phosphatidylcholine.

3-4. (canceled)

5. The particle of claim 1, wherein the liposome further comprises a third lipid; and the third lipid is a sterol selected from the group consisting of cholesterol, campesterol, desmosterol, stigmasterol, lanosterol, and sitosterol.

6-7. (canceled)

8. The particle of claim 1, wherein the liposome further comprises a fourth lipid; and the fourth lipid is an anionic lipid selected from the group consisting of 1-palmitoyl-2-oleoyl-sn-glycero-3-phospho-(1′-rac-glycerol) (POPG), 1,2-distearoyl-sn-glycero-3-phosphoglycerol (DSPG), 1,2-dipalmitoyl-sn-glycero-3-phosphoglycerol (DPPG), 1,2-dioleoyl-sn-glycero-3-phospho-rac-(1′-glycerol) (DOPG), 1-palmitoyl-2-oleoyl-sn-glycero-3-phosphoinositol (POPI), 1,2-distearoyl-sn-glycero-3-phosphoinositol (DSPI), 1,2-dioleoyl-sn-glycero-3-phospho-(1′-myo-inositol) (DOPI), egg phosphatidylglycerol, and soy phosphatidylglycerol.

9-11. (canceled)

12. The particle of claim 1, wherein at least one first lipid is a phospholipid selected from the group consisting of 1,2-dioleoyl-sn-glycero-3-phosphoethanolamine-N-[4-(p-maleimidophenyl) butyramide] (MPB-PE), 1,2-dioleoyl-sn-glycero-3-phosphoethanolamine-maleimide (DOPE-Mal), 1,2-dioleoyl-sn-glycero-3-phosphoethanolamine-N-hydroxysuccinimide (DOPE-NHS), 1,2-dioleoyl-sn-glycero-3-phosphoethanolamine-azide (DOPE-azide), 1,2-dioleoyl-sn-glycero-3-phosphoethanolamine-dibenzocyclooctyne (DOPE-DBCO), and 1,2-dioleoyl-sn-glycero-3-phosphoethanolamine-trans-cyclooctene (DOPE-TCO).

13. (canceled)

14. The particle of claim 1, wherein the liposome comprises 25-90% DSPC, 0-45% cholesterol, 3-30% POPG, and 1-10% MPB-PE.

15. The particle of claim 14, wherein the liposome comprises 65% DSPC, 24% cholesterol, 6% POPG, and 5% MPB-PE.

16-19. (canceled)

20. The particle of claim 1, wherein the first lipid is covalently bonded to the immunostimulatory agent via maleimide-cysteine conjugation, N-hydroxysuccinimide easter-amine crosslinking, click chemistry, Staudinger ligation, Diels-alder cycloaddition, photoinducible ligation, oxime-hydrazone ligation, tetrazine ligation, palladium-ruthenium catalysis, disulfide formation, or Michael addition.

21. (canceled)

22. The particle of claim 1, wherein the polycation is selected from the group consisting of poly-L-arginine (PLR), poly-L-lysine (PLL), polyethylenimine (PEI), poly(diallyldimethylammonium chloride) (PDAC), poly(β-amino esters) (PBAE), and poly(allylamine hydrochloride) (PAH), or a combination or derivative thereof.

23. (canceled)

24. The particle of claim 1, wherein the polyanion is selected from the group consisting of poly-L-glutamic acid (PLE), poly-L-aspartic acid (PLD), poly-hyaluronic acid (HA), poly-acrylic acid (PAA), and poly(sodium 4-styrenesulfonate), or a combination or derivative thereof.

25-26. (canceled)

27. The particle of claim 1, wherein the at least one immunostimulatory agent is selected from the group consisting of a cytokine, an antibody, an affibody, a nanobody, a protein, a peptide, and variants of the foregoing.

28-29. (canceled)

30. The particle of claim 27, wherein the cytokine is selected from the group consisting of interleukin-12 (IL-12), single chain interleukin 12 (scIL-12), interleukin-2 (IL-2), interferon-γ (IFN-γ), interferon-α (IFN-α), interferon-β (IFN-β), interleukin-15 (IL-15), interleukin-15 super agonist (IL-15SA), interleukin-18 (IL-18), tumor necrosis factor alpha (TNF-α), interleukin-10 (IL-10), interleukin-8 (IL-8), TNF-related apoptosis-inducing ligand (TRAIL), FMS-like tyrosine kinase 3 ligand (FLT3LG), and variants thereof.

31-42. (canceled)

43. A pharmaceutical composition comprising a plurality of particles of claim 1, and an a pharmaceutically acceptable excipient.

44. The pharmaceutical composition of claim 43, further comprising an additional pharmaceutical agent selected from the group consisting of a chemotherapeutic, targeted therapy, gene therapy, immune therapy, and hormone therapy.

45-46. (canceled)

47. The pharmaceutical composition of claim 44, wherein the immune therapy is an immune checkpoint therapy selected from the group consisting of anti-PD1 immune checkpoint therapy, anti-PDL1 immune checkpoint therapy, anti-CTLA4 immune checkpoint therapy, anti-TIM-3 immune checkpoint therapy, anti-LAG-3 immune checkpoint therapy, anti-NKG2A immune checkpoint therapy, anti-CD73 immune checkpoint therapy, anti-A2aR immune checkpoint therapy, anti-B7-H3 immune checkpoint therapy, and anti-B7-H4 immune checkpoint therapy, or a combination thereof.

48-55. (canceled)

56. A method of delivering an immunostimulatory agent to a target cell, wherein the method comprises contacting the target cell with a particle of claim 1.

57. A method of recruiting immune cells to a target cell, wherein the method comprises contacting the target cell with a particle of claim 1.

58-66. (canceled)

67. A method of treating a disease in a subject in need thereof, the method comprising administering to the subject an effective amount of a particle of claim 1.

68-71. (canceled)

72. The method of claim 67, wherein the disease is selected from the group consisting of colon cancer, ovarian cancer, breast cancer, head and neck cancer, lung cancer, and brain cancer.

73-76. (canceled)

77. A kit comprising:
a particle of claim 1; and
instructions for using the particle.

78. The particle of claim 30, wherein the cytokine is scIL-12 comprising a C-terminal cysteine.
